(19) United States
(12) Patent Application Publication
(10) Pub. No.: US 2002/0183290 A1
(43) Pub. Date: Dec. 5, 2002

(54) Adrenergic receptor antagonists selective for both alpha1A-and alpha1D-subtypes and uses therefor

(75) Inventors: Amedeo Leonardi, Milan (IT); Gianni Motta, Barlassina (IT); Rodolfo Testa, Vignate (IT)

Correspondence Address: DARBY & DARBY P.C., 805 Third Avenue, New York, NY, 10022, US

(73) Assignee: Recordati S.A., Chemical and Pharmaceutical Company

(21) Appl. No.: 10/060,925

(22) Filed: Jan. 30, 2002

Related U.S. Application Data
(60) Provisional application No. 60/311,389, filed on Aug. 10, 2001.

(30) Foreign Application Priority Data
Jan. 30, 2001 (IT) MI 2001A 000164

(51) Int. Cl. A61K 31/56

(52) U.S. Cl. 514/169

(57) ABSTRACT

Described are derivatives with an adrenergic antagonistic activity and, in particular, high selectivity for α₁ₐ- and α_{1d}-adrenergic receptors compared to α_{1b}-receptors. This selectivity profile suggests the use of these derivatives in the treatment of symptoms of the lower urinary tract, including those associated to benign prostatic hyperplasia, without the side effects associated with hypotensive activity.

This application claims priority under 35 U.S.C. §119(e) of provisional patent application serial No. 60/311,389, filed Aug. 10, 2001, and priority under 35 U.S.C. §119(a)-(d) of Italian patent application MI 2001A 000164, filed Jan. 30, 2001. Each of the aforesaid applications is hereby incorporated herein by reference in its entirety.

### SCOPE OF THE INVENTION

This invention relates to α₁-adrenergic antagonists that are selective ligands of the α₁ₐ and α_{1d} subtypes, to pharmaceutical compositions containing them and to uses for such selective ligands and compositions containing them.

### BACKGROUND OF THE INVENTION

Lower urinary tract symptoms (LUTS) resulting from bladder-neck obstruction (BNO) is a common disorder in urology. The etiology of LUTS can be secondary to anatomical or functional causes, or a combination of these causes.

Causes of BNO include prostatic enlargement (benign or malignant), bladder neck contracture, urethral stricture, and meatal stricture. Symptoms associated with BNO are classified as obstructive or irritative. Obstructive symptoms include hesitancy, poor stream, prolonged urination and feelings of incomplete emptying. Irritative symptoms consist of frequency, urgency, nocturia and unstable-bladder contractions.

The bladder is functionally and anatomically divided into the detrusor (body and ventral base) and trigone (dorsal portion of base extending between the ureteral orifices and the bladder neck). The detrusor and trigone differ in their histological, histochemical and pharmacological properties. In contrast, the trigone and prostate have similar vascular supply and innervation, and express similar receptors.

LUTS can occur secondarily to benign prostatic hypertrophy (BPH).

BPH is a progressive condition that is characterized by a nodular enlargement of both glandular (epithelial) and stromal (fibromuscular) prostatic tissue, resulting in obstruction of the urethra. The increase in stromal mass is the key factor in the pathogenesis of clinically significant BPH. The symptoms of BPH include increased frequency of urination, nocturia, a poor urinary stream and hesitancy or delay in initiating urine flow. The physiology of BPH has two components: (1) a static component related to the increase in prostatic cellular mass and (2) a dynamic component related to variations in prostatic smooth muscle tone (Caine et al., 1975, Brit. J. Urol 47:193-202).

Chronic consequences of BPH can include hypertrophy of bladder smooth muscle and a decompensated bladder, which may lead to LUTS, and an increased incidence of urinary tract infection. The specific biochemical, histological and pharmacological properties of the prostate adenoma leading to BNO are not yet known. However, the development of BPH is considered to be an inescapable phenomenon for the ageing male population. BPH is observed in approximately 70% of males over the age of 70. Currently, the specific method of choice for treating BPH is surgery. A pharmacological alternative to surgery is clearly very desirable. The limitations of surgery for treating BPH include the morbidity rate of an operative procedure in elderly men, persistence or recurrence of obstructive and irritative symptoms, as well as the significant cost of surgery.

Much attention has been focused on the role of the sympathetic nervous system and α₁-adrenergic receptors in the dynamic component of BNO. Clinical studies have found that α₁-adrenergic antagonists relax prostatic smooth muscle, relieving obstructive symptoms (Caine, 1990, Urol. Clin. N. Am. 17:641-649; Lepor et al., 1992, J Urol., 148:1467-1474). α-Adrenergic receptors (McGrath et al., 1989, Med. Res. Rev. 9:407-533) are specific neuroreceptor proteins located in the peripheral and central nervous systems on tissues and organs throughout the body. These receptors are important targets for controlling many physiological functions and, thus, represent important objectives for drug development. In fact, many α adrenergic drugs have been developed over the past 40 years. Examples include clonidine, phenoxybenzamine and prazosin, terazosin, alfuzosin, doxazosin, tamsulosin (treatment of hypertension), naphazoline (nasal decongestant), and apraclonidine (treating glaucoma). α-Adrenergic drugs can be broken down into two distinct classes: agonists (e.g., clonidine and naphazoline), which mimic the receptor activation properties of the endogenous neurotransmitter noradrenaline, and antagonists (e.g., phenoxybenzamine and prazosin, terazosin, alfuzosin, doxazosin and tamsulosin), which act to block the effects of noradrenaline. Many of these drugs are effective, but also produce unwanted side effects (clonidine, for example, produces dry mouth and sedation in addition to its antihypertensive effect).

The above reported agonists are selective for the α₂-adrenergic receptor whereas most antagonists are selective for the α₁-adrenergic receptor, with the exception of tamsulosin which shows a considerable affinity also for the 5-HT_{1A} receptor. Many of the cited α₁ antagonists are currently used for the therapy of BPH but, due to their poor uroselectivity, they are liable to cause undesirable cardiovascular side effects.

Recent pharmacological, biochemical and radioligand-binding studies have lead to the description of three different α₁-receptor subtypes with a high affinity for prazosin, namely α_{1A}-(α₁ₐ-), α_{1B}-(α_{1b}-) and α_{1D}-(α_{1d}-) subtypes, with lower case subscripts being used for recombinant receptors and upper case subscripts for receptors in native tissues (Hieble et al., 1995, Pharmacol. Rev., 47: 267-270). In functional studies, α₁ receptors with a low affinity for prazosin have also been identified and termed α_{1L} receptors (Flavahan et al, 1986, Trends Pharmacol. Sci., 7: 347-349; Muramatsu et al., 1995, Pharmacol. Comm., 6:23-28).

Several studies have demonstrated the presence of these α₁-adrenergic subtypes in the lower-urinary-tract tissues (Andersson K. E., “4^{th} International Consultation in Benign Prostatic Hyperplasia (BPH)”, Paris, Jul. 2-5, 1997, pp. 601-609).

Several other studies have shown that the human prostate receives innervation from both the sympathetic and parasympathetic nervous systems.

The adrenergic nerves, however, are considered responsible for prostatic smooth muscle tone by releasing noradrenaline, thus stimulating contraction-mediating α₁-adrenoceptors. Approximately 50% of the total urethral pressure in BPH patients may be due to α₁-adrenoceptor-mediated muscle tone. Functional studies have indicated the occurrence of important adrenoceptor functions in prostatic adenomatous and capsular tissue. Clinical studies with the prototypical non-selective α₁-adrenoceptor antagonist, prazosin, reinforced the key role of α₁, adrenoceptors in the control of prostatic smooth-muscle tone. This was also confirmed in the laboratory by studies showing that, although both α₁- and α₂-adrenergic receptors are present within the human prostate, the contractile properties are mediated primarily by α₁-adrenergic receptors. Many clinical investigations have confirmed that α₁-adrenoceptor blockade relieves lower-urinary-tract symptoms (LUTS), both of irritative and obstructive type, in patients with BPH.

Separate subtypes of α₁-adrenergic receptors, a group (α_{1H}) with a high and a group (α_{1L}) with a low affinity for prazosin, have been suggested to be present in the human prostate. All three high-affinity α₁-adrenoceptor subtypes found in molecular cloning studies have been identified in prostatic stromal tissue. The α₁ₐ-subtype was found to be dominant, representing about 60-85% of the α₁-adrenoceptor population. Recent findings suggest that there may be quantitative differences in subtype populations between normal and hyperplastic prostates, the ratios between the subtypes a₁ₐ:α_{1b}:α_{1d} being 85:1:14 in BPH tissue and 63:6:31 in non-BPH tissue.

The α_{1A}-adrenergic receptor was reported to mediate the contractile response of the human prostate in vitro. Ford et al. (1995, Br. J Pharmacol. 114:24 P) observed that the α_{1A}-adrenergic receptor may not mediate contractile responses to noradrenaline, and suggested the α_{1L}-adrenergic receptor as a candidate. Findings by Kenny et al. (1996, Br. J Pharmacol. 118:871-878) supported the view that the α_{1L}-adrenergic receptor, which appears to share many of the characteristics of an α_{1A}-adrenergic receptor, mediates the contractile response of the human prostate. Other data suggests, however, that the α_{1L}-and α_{1A}-adrenergic receptors may represent separate affinity states of the same receptor (Ford et al., 1997, Br. J Pharmacol. 121:1127-1135). Therefore, it is now confirmed that the α₁ₐ subtype is the subtype important in mediating prostate smooth muscle contraction.

The fact that α₁ₐ-adrenoceptor predominates in prostate smooth muscle suggested a safer use of α₁ₐ-selective antagonists to treat LUTS secondary to BPH. However, a clinical trial performed with the selective antagonist of the α₁ₐ-adrenergic receptor, Rec 15/2739, did not result in relief of LUTS, despite the presence of relaxing effects on prostate smooth muscle (Hieble et al., 1996, Pharmacol. Res. 33:145-160). Thus, this important finding indicated that relieving obstructive conditions is not sufficient to significantly relieve LUTS.

LUTS also develop in women as they age. As in men, LUTS in women includes both filling symptoms such as urgency, incontinence, and nocturia, and voiding symptoms, such as weak stream, hesitancy, intermittency, incomplete bladder emptying and abdominal straining. The presence of LUTS in both men and women suggests that at least part of the underlying etiology may be similar in the two sexes.

In a recent study, an α₁-antagonist was reported to reduce LUTS in women more effectively than an anticholinergic (Serels et al., 1998, Neurology and Urodynamics 17:31-36). The authors suggested that there appeared to be a role for α₁ antagonists in treating LUTS in women. The possible causes of the conditions which can explain these results are: a) dysfunction of the bladder neck and urethra, causing functional BNO (analogous to BPH-induced BNO) causing detrusor overactivity; and b) increased α₁-adrenoreceptor activity in the detrusor, causing frequency and urgency. On these bases, α₁ antagonists are used in clinical practice to treat LUTS in women as well as men.

The results of Serels et al. also indicated that the combined administration of α₁ antagonists and anticholinergics can have improved efficacy in treatment of LUTS, as suggested also by Fitzpatrick (2000, International British J. Urol. 85, Supp. 2:1-5).

The finding that non-selective α₁ antagonists are useful in treating LUTS of both prostatic and non-prostatic origin in both males and females shows the usefulness of these compounds in treating LUTS of both obstructive and non-obstructive origins, in males as well as females.

These results suggested that LUTS involves more organs than simply the prostate, and justified further studies to search for α₁-adrenergic receptors in non-prostatic tissue. mRNA for the α₁ receptor was found in the female urethra, with autoradiography confirming the predominance of the α_{1A} subtype (Andersson K. E., 2000, Brit. J Urol. Intl. 85, Supp. 2:12-18).

The α_{1A} subtype also predominated in prostate and bladder trigone and was shown to be essential in mediating contraction in these tissues. (Price et al., 1993, J Urol. 150:546-551; Chapple, 1998, Eur. Urol., 34(Suppl. 1): 10-17; Forray et al., 1994, Mol. Pharmacol. 45:703-708; and Lepor et al., 1994, J. Pharmacol. Expt. Ther. 270:722-777). Both α₁ₐ and α_{1d} subtypes were found in the human detrusor, with the α_{1d} subtype predominant (Malloy et al., 1998, J. Urol 160: 937-943).

International application PCT/US99/09846 (published as WO 99/57131) of Schwinn discloses the use of α_{1d}-selective antagonists in the treatment of LUTS without the side effects of non-selective α₁ antagonists. Selectivity is therein defined as at least two-fold selectivity for α_{1d} relative to α₁ₐ or α_{1b} Schwinn also discloses the use of α₁ antagonists that bind selectivity to both α₁ₐ and α_{1d} subtypes relative to α_{1b} subtypes. No guidance was provided, however, as to the relative affinity for α₁ₐ versus α_{1d} subtypes. Nor was any guidance provided on how to prepare or use compounds that are selective for α₁ₐ and α_{1d} subtypes relative to the α_{1b} subtype.

Abrams et al (1995, Br. J. Urol., 76:325-336) disclosed the use of tamsulosin to treat patients with BPH. The same author ascribed the efficacy of tamsulosin in treating LUTS to this molecule's capacity to interact with α₁ₐ-adrenergic receptors.

There is thus a need for methods of treating LUTS without the side effects of non-selective α₁-adrenergic antagonists. In particular, there remains a need for identifying selective antagonists of α₁-adrenoceptor subtypes.

We have tested α₁-antagonists for selectivity in binding α₁ₐ-, α_{1b}- and α_{1d}-subtypes. Furthermore, using an animal model that reflects BNO effects in humans to test the effects of selective and non-selective α₁-antagonists on bladder function, we have found that antagonists that are selective for α₁ₐ- and α_{1d} -subtypes relative to the α_{1b}-subtype are more effective inhibitors of unstable-bladder contractions, compared to antagonists that are selective for a single subtype.

On these bases, antagonists that are selective for the combination of α₁ₐ- and α_{1d}-subtypes relative to the α_{1b}-subtype can be an effective means to treat lower urinary-tract disorders.

Another possible use of α₁-antagonists is the management of neurogenic lower urinary tract dysfunction (NLUTD), caused by neurological disease or trauma. NLUTD may lead to debilitating symptoms and serious complications, including increased urinary frequency, incontinence, voiding difficulty, recurrent upper-urinary-tract infections, and upper-urinary-tract deterioration. Management of NLUTD is indicated to preserve renal function and avoid urological complications. Administration of α₁-antagonists may benefit patients with NLUTD by facilitating bladder filling by alleviating high detrusor pressure during bladder filling, which is evidenced by poor bladder compliance and detrusor hyperreflexia. In both animal models and patients with spinal cord injury resistant to anticholinergics, α₁-antagonists improved bladder compliance. (Serels, ibid.; Fitzpatrick, ibid.; Kakizaki et al., 2000, Brit. J. Urol International 85, Supp. 2: 25-30; Sundin et al., 1977, Invest. Urol. 14: 322-328; McGuire et al., 1985, Neurology and Urodynamics 4:139-142; Swierzewski et al., 1994, J Urol. 151: 951-954).

### SUMMARY OF THE INVENTION

The invention discloses compounds of general formula I:

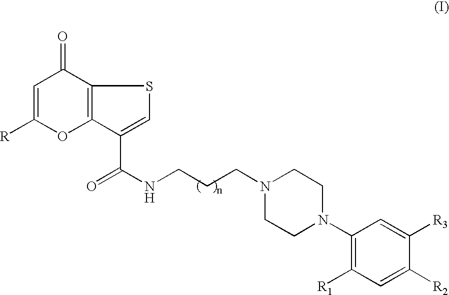

where

R is an aryl, cycloalkyl or polyhaloalkyl group,

R₁ is chosen from the group consisting of alkyl, alkoxy, polyfluoroalkoxy, hydroxy and trifluoromethanesulfonyloxy groups;

each of R₂ and R₃ independently represents a hydrogen atom, or a halogen, or an alkoxy or polyfluoroalkoxy group,

and n is 0, 1 or 2.

Without limitations, the preferred aryl group which R may represent is phenyl; the preferred cycloalkyl group that R may represent is cyclohexyl; the preferred polyhaloalkyl group that R may represent is trifluoromethyl. The preferred alkyl group which R₁ may represent without limitation is C₁₋₄ lower alkyl. Preferred alkoxy groups which R₁, R₂, and R₃ may represent without limitation are lower C₁₋₄ alkoxy groups, most preferably methoxy. Preferred polyfluoroalkoxy which R₁, R₂, and R₃ may represent without limitation are trifluoromethoxy or 2,2,2-trifluoroethoxy.

The preferred value for n is 1.

Also preferred is where R₁ is chosen from the group consisting of alkoxy and hydroxy; R₂ is chosen from the group consisting of hydrogen and halogen; R₃ is chosen from the group consisting of hydrogen and halogen; and n is 0, 1 or 2.

Also preferred is where R₁ is chosen from a group consisting of alkoxy, hydroxy and polyfluoroalkoxy; R₂ is chosen from the group consisting of hydrogen, halogen and alkoxy; R₃ is chosen from the group consisting of hydrogen, halogen and alkoxy; and n is 0, 1 or 2.

Also preferred is where R₁ is chosen from the group consisting of alkoxy, polyfluoroalkoxy, hydroxy; R₂ is halogen; R₃ is hydrogen; and n is 0, 1 or 2.

The invention also includes the N-oxides and pharmaceutically acceptable salts of these compounds.

The invention further provides pharmaceutical compositions comprising a compound of Formula I or a N-oxide or pharmaceutically acceptable salt of such a compound in admixture with a pharmaceutically acceptable diluent or carrier.

In another aspect the invention provides compounds of general formula II:

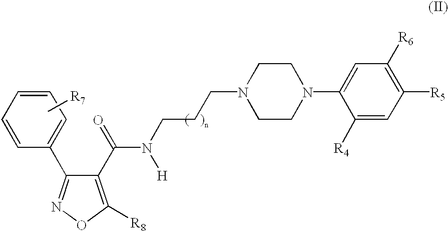

wherein:

R₄is selected from the group consisting of alkyl, alkoxy, polyfluoroalkoxy, hydroxy and trifluoromethanesulfonyloxy group;

each of R₅ and R₆ independently is selected from the group consisting of hydrogen atom, halogen atom, polyfluoroalkoxy and alkoxy groups;

R₇ represents one or more substituents selected from the group consisting of hydrogen atom, halogen atom, alkyl, alkoxy, nitro, amino, acylamino, cyano, alkoxycarbonyl and carboxamido group;

R₈ is selected from the group consisting of a hydrogen atom, an alkyl group and an arylalkyl group; and

n is 0, 1 or 2.

The invention also includes the N-oxides and pharmaceutically acceptable salts of these compounds.

Preferred alkyl groups which R₄ and R₈ may represent are without limitation lower (C₁₋₄) alkyl groups, preferably methyl. Preferred alkoxy groups which R₄, R₅, R₆ and R₇ may represent without limitation are lower (C₁₋₄) alkoxy groups, preferably methoxy. Preferred polyfluoroalkoxy groups which R₄, R₅ and R₆ may represent are without limitation trifluoromethoxy or 2,2,2-trifluoroethoxy groups. The preferred value for n is 1.

A preferred arylalkyl group that R₈ may represent without limitation is phenylalkyl, optionally substituted with one or more substituent selected from the group consisting of hydrogen, halogen, C₁₋₄ alkyl, alkoxy, nitro, amino, acylamino, cyano, alkoxycarbonyl and carboxamido group.

A preferred group that R₇ may represent without limitation is carboxamido.

Also preferred is where R₄is selected from the group consisting of alkoxy and hydroxy; R₅ is selected from the group consisting of hydrogen and halogen; R₆ is selected from the group consisting of hydrogen and halogen; R₇ represents one or more substitutents consisting of hydrogen, halogen, alkyl, alkoxy, nitro, amino, acylamino, cyano, alkoxycarbonyl and carboxamido group; R₈ is selected from the group consisting of hydrogen and alkyl; and n is 0, 1 or 2.

Also preferred is where R is selected from a group consisting of alkoxy, alkyl and polyfluoroalkoxy; R₅ is selected from the group consisting of hydrogen, halogen and alkoxy; R₆ is selected from the group consisting of hydrogen, halogen and alkoxy; R₇ represents one or more substitutents selected from the group consisting of hydrogen, halogen, alkyl, alkoxy, and amino; R₈ is selected from the group consisting of hydrogen and alkyl; and n is 0, 1 or 2.

The compounds of the invention include those compounds where, independently, R₄ is selected from the group consisting of methyl, methoxy, 2,2,2,-trifluoroethoxy, hydroxy and trifluoromethanesulfonyloxy, R₅ is selected from a group consisting of hydrogen and fluorine, R₆ is selected from the group consisting of a hydrogen, chlorine and 2,2,2-trifluoroethoxy, R₇ is carboxamido, R₈ is selected from the group consisting of methyl, ethyl, 2-phenylethyl, 3-phenylpropyl and hydrogen; and n is 0, 1 or 2.

In yet another aspect the invention provides compounds of general formula III:

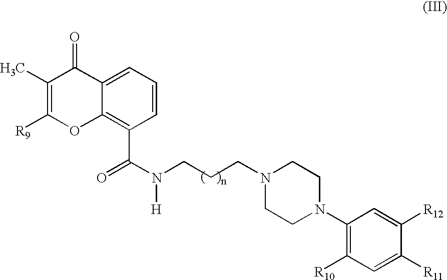

wherein

R₉ is selected from the group consisting of a phenyl, alkoxycarbonyl, alkylcarbonyl, carbamoyl, alkylcarbamoyl, dialkylcarbamoyl, cyano and alkoxycarbonylamino group;

R₁₀ is selected from the group consisting of an alkyl, alkoxy, polyfluoroalkoxy, hydroxy and trifluoromethanesulphonyloxy group;

each of R₁₁ and R₁₂ independently is selected from the group consisting of hydrogen atom, halogen atom, polyfluoroalkyl, polyfluoroalkoxy, cyano and carbamoyl group; and

n is 0, 1 or 2;

with the proviso that, if R₉ represents a phenyl group and both R₁₁ and R₁₂ represent hydrogen and/or halogen atoms, then R₁₀ represents a polyfluoroalkoxy or trifluoromethanesulphonyloxy group.

The invention also includes the N-oxides and pharmaceutically acceptable salts of these compounds.

When R₉ does not represent a phenyl group, each of R₁₁ and R₁₂ preferably and independently represents a hydrogen or halogen atom or a polyfluoroalkoxy group.

Alkyl and alkoxy groups preferably have from 1 to 4 carbon atoms; complex groups such as alkoxycarbonyl, alkylcarbonyl, alkylcarbamoyl, dialkylcarbamoyl, polyfluoroalkyl, polyfluoroalkoxy and alkoxycarbonylamino, are preferably construed accordingly. Preferred polyfluoroalkoxy groups are trifluoromethoxy and 2,2,2-trifluoroethoxy. The preferred value for n is 1.

Further preferred is where R₉ is selected from the group consisting of phenyl, alkoxycarbonyl, alkylcarbonyl, carbamoyl, alkylcarbamoyl, dialkylcarbamoyl, cyano and alkoxycarbonylamino; R₁₀ is selected from the group consisting of alkoxy and hydroxy; each of R₁₁ and R₁₂ is independently selected from the group consisting of hydrogen, halogen, polyfluoroalkyl and carbamoyl; and n=0, 1 or 2.

Further preferred is where R₉ is selected from the group consisting of carbamoyl, alkylcarbamoyl and dialkylcarbamoyl; R₁₀ is selected from the group consisting of alkoxy, polyfluroalkoxy, hydroxy and trifluoromethanesulphonyloxy; each R₁₁ and R₁₂ is independently selected from the group consisting of hydrogen, halogen, polyfluoroalkyl, polyfluoroalkoxy, cyano and carbamoyl; and n=0, 1 or 2.

Further preferred is where R₉ is carbomoyl; R₁₀ is selected from the group consisting of alkoxy; polyfluoroalkoxy, hydroxy, and trifluoromethanesulphonyloxy; each R₁₁ and R₁₂ is independently selected from the group consisting of hydrogen, halogen, polyfluoroalkyl, polyfluoroalkoxy, cyano and carbamoyl; and n=0, 1 or 2.

The compounds of the invention include those compounds where, independently, R₉ is selected from the group consisting of an alkoxycarbonyl, alkylcarbonyl, carbamoyl, alkylcarbamoyl, dialkylcarbamoyl, cyano and alkoxycarbonylamino group, R₁₀ is selected from a group consisting of methyl, methoxy, trifluoromethoxy, 2,2,2-trifluoroethoxy, hydroxy and trifluoromethanesulphonyloxy group, R₁₁ is selected from the group consisting of hydrogen and fluorine, R₁₂ is selected from the group consisting of hydrogen, fluorine, chlorine, trifluoromethyl, 2,2,2-trifluoroethoxy, cyano and carbamoyl; and n is 0, 1 or 2.

The invention further provides pharmaceutical compositions comprising a compound of the general formula I, II or III, or a N-oxide or pharmaceutically acceptable salt of such a compound in admixture with a pharmaceutically acceptable diluent or carrier. A preferred N-oxide is a piperazine-N-oxide, which may be formed at either nitrogen atom of a piperazine ring. Preferences are as outlined above for the compounds of the invention.

In another aspect, the present invention is directed to methods for preventing contractions (including noradrenaline-mediated contractions) of the urethra, bladder and other organs of the lower urinary tract without substantially affecting blood pressure, by administering a compound that binds selectively to α₁ₐ- and α_{1d}-adrenergic receptors relative to the α_{1b} adrenergic receptor and has a structure as given by general formulas I, II or III to a mammal in need of such treatment in an amount or amounts effective for the particular use. In a preferred embodiment, said mammal is a human.

In yet another aspect, the invention is directed to methods for blocking α₁ receptors by exposing said receptors (e.g., by delivery to the environment of said receptors, by addition to an extracellular medium, or by administering to a mammal possessing said receptors) an effective amount of a compound of the invention, in this way relieving diseases associated to overactivity of said receptors that can be treated with α₁ antagonists.

Other aspects of the invention are methods of treatment using antagonists of α₁ₐ- and α_{1d} adrenergic receptors for lowering intraocular pressure, inhibiting cholesterol biosynthesis, treating cardiac arrhythmia and sexual dysfunction, including erectile dysfunction, and relieving pain of a sympathetic origin. The methods of treatment comprise administering an effective amount of a selective α₁-adrenergic antagonist of the present invention or a pharmaceutical composition thereof to a patient in need of such treatment.

It is understood that “of a sympathetic origin” is defined as any physiological sensation, condition or response that depends upon any component of the sympathetic nervous system, can be modulated by the action of any component of the sympathetic nervous system, or can be affected by treatment of any component of the sympathetic nervous system.

A further object of the present invention is the release of the selective antagonists of the α₁ₐ and α_{1d}-adrenergic receptors of present invention or pharmaceutical compositions containing them in the environment of α₁-adrenergic receptors wherein said release is effected by administering compounds of the present invention or pharmaceutical compositions containing them to a mammal, including a human, possessing said receptors.

A further object of the present invention is a method of treatment of a patient suffering from BPH, the method comprising administering an effective amount of a selective α₁-adrenergic antagonist of the present invention or a pharmaceutical composition containing it to a patient in need of such treatment.

A further object of the present invention is the method for the treatment of lower-urinary-tract symptoms (LUTS), which include but are not limited to filling symptoms, urgency, incontinence and nocturia, as well as voiding problems such as weak stream, hesitancy, intermittency, incomplete bladder emptying and abdominal straining, the method comprising administering an effective amount of a selective α₁-adrenergic antagonist of the present invention or a pharmaceutical composition containing it to a patient in need of such treatment, and further comprising the possibility of concurrently administering an anticholinergic compound which may be selected from the group consisting of tolterodine, oxybutinin, darifenacin, alvameline and temiverine.

A further object of the present invention is the method for the treatment of neurogenic lower urinary tract dysfunction (NLUTD), the method comprising administering an effective amount of a selective α₁-adrenergic antagonist of the present invention or a pharmaceutical composition containing it to a patient in need of such treatment and further comprising the possibility of concurrently administering an anticholinergic compound which may be selected from the group consisting of tolterodine, oxybutinin, darifenacin, alvameline and temiverine.

A further object of the present invention is the treatment of LUTS in females, which includes but is not limited to, filling symptoms, urgency, incontinence and nocturia as well as voiding problems such as weak stream, hesitancy, intermittency, incomplete bladder emptying, and abdominal straining, the method comprising administering an effective amount of a selective α₁-adrenergic antagonist of the present invention or a pharmaceutical composition containing it to a woman in need of such treatment, and further comprising the possibility of concurrently administering an anticholinergic compound which may be selected from the group consisting of tolterodine, oxybutinin, darifenacin, alvameline and temiverine

Other features and advantages of the present invention will be apparent to those skilled in the art from the following detailed description and claims.

### DETAILED DESCRIPTION OF THE INVENTION

All patents, patent applications and references cited herein are hereby incorporated by reference in their entirety. In the case of inconsistencies in definitions, the to present description will control.

It is further understood that all compounds described, listed and represented herein are meant to include all hydrates, solvates, polymorphs and pharmaceutically-acceptable salts thereof.

Some of the compounds described herein contain one or more asymmetric centers and may thus give rise to diastereomers and optical isomers. The present invention is meant to comprehend such possible diastereomers as well as their racemic and resolved, enantiomerically-pure forms and pharmaceutically acceptable salts thereof.

Some of the compounds described herein contain olefinic double bonds, and unless specified otherwise, are meant to include both E and Z geometric isomers.

The invention provides methods for treatment of lower-urinary-tract symptoms (LUTS), particularly those involving micturition, such as dysuria, incontinence, and enuresis. Said methods involve administering to patients selective antagonists of the α₁ₐ and α_{1d}-subtypes of adrenergic receptors, relative to the α_{1b}-subtype of adrenergic receptor, for a sufficient time and in an amount effective for relieving or ameliorating at least one symptom of the micturition disorders. Such symptoms include but are not limited to filling symptoms, urgency, incontinence and nocturia, as well as voiding problems such as weak stream, hesitancy, intermittency, incomplete bladder emptying and abdominal straining.

The term “mammal” includes humans.

The term “treatment” is defined as the prevention, disappearance, or amelioration of at least one of the foregoing LUTS.

“Obstructive symptoms” typically include hesitancy, poor stream, prolonged urination and feelings of incomplete emptying.

“Irritative symptoms” typically include frequency, urgency, nocturia and unstable bladder contractions.

The invention provides for treatment of both obstructive and irritative symptoms of the lower urinary tract. In a preferred aspect the invention provides for treatment of irritative symptoms due to bladder-neck obstruction (BNO) that may be secondary to obstructive disorders such as, for example, BPH.

Efficacy of treatment may be determined by any known method. Such methods include but are not limited to determining urination volumes, frequency of urination, and frequency and strength of bladder contractions in individuals with neuromuscular dysfunction of the lower urinary tract; or and interviewing such individuals to determine if they have experienced the amelioration of any of these symptoms. Other measures of efficacy include a measurable reduction, most preferably a clinically relevant reduction, of urine leakage related to feelings of urgency, urine leakage related to physical activity, coughing or sneezing, leakage of small amounts, e.g., drops of urine, difficulty in bladder emptying, urine leakage not related to urgency or activity, nocturia, bedwetting, a feeling of incomplete bladder emptying, etc.

The use of questionnaires and scales to measure symptom severity is widely accepted, complementing objective clinical measures and having the advantage of being inexpensive and potentially self-administered.

Female and male lower-urinary-tract questionnaires are available which provide a method of measuring symptom severity and life quality in a reproducible and valid fashion and allow an exact description of specific lower-urinary-tract symptoms.

The sums of scores collected for the questions included in the questionnaires are highly correlated with patients' ratings of the magnitude of their urinary problems, and are very sensitive to changes induced by treatment (Jackson et al., 1996, Brit. J. Urol., 77:805-812).

The adrenergic antagonistic activity of the compounds of the invention renders them useful as agents acting on body tissues particularly rich in α₁ adrenergic receptors (such as prostate, urethra and bladder). Accordingly, the selective adrenergic antagonists within the invention, established as such on the basis of their receptor-activity profile, can be useful therapeutic agents for the treatment of, for example, micturition problems associated with obstructive disorders of the lower urinary tract, including, but not limited to, BPH.

The α₁-adrenergic antagonistic drugs currently used for the symptomatic therapy of BPH are poorly selective for α₁-adrenergic subtypes and are thus subject to cause relevant side effects due to their action on the cardiovasular systems.

The α₁ₐ- and α_{1d}-selective antagonists suitable for use in practicing the present invention include, without limitation, those compounds having one or more of the following properties:

(1) Significant affinity for the α₁ₐ- and α₁-subtypes of α₁-adrenergic receptors: Useful compounds preferably bind to the α₁ₐ- and α_{1d}-subtypes of α₁-adrenergic receptors with an affinity of between about 100 and 0.1 nM. Without limitations, as described in detail below, affinity may be measured by determining the Ki of compounds in vivo or in vitro, in cell extracts or fractions of extracts. Ki can be determined using, for example, native or recombinant α₁-adrenergic receptors and receptors that have expressed in native or non-native species and/or cell types.

(2) Selectivity: Compounds of the invention exhibit at least about 10-fold greater affinity for a α₁ₐ receptors relative to α_{1b} receptors, and at least about 6-fold greater affinity for α_{1d} receptors relative to α_{1b} receptors. More preferred are compounds that exhibit about 10-fold greater affinity for both α₁ₐ and α_{1d} receptors relative to α_{1b} receptors. Most preferred are compounds that exhibit about 20-fold greater affinity for both α₁ₐ and α_{1d} receptors relative to the α_{1b} receptors. In all cases, the compounds of the invention bind to the α₁ₐ and α_{1d} receptors with affinities that are within 10-fold of each other.

Compounds belonging to the general class defined above are thus suitable for screening to identify compounds useful in treating lower urinary-tract-symptoms and are exemplified, without limitation, by:

Compound A: N-{3-[4-(2-methoxyphenyl)-1-piperazinyl]propyl}-7-keto-5-trifluoromethyl-7H-thieno [3,2-b]pyran-3-carboxamide;

Compound B: N-{3-[4-(5-chloro-2-methoxyphenyl) -1-piperazinyl]propyl}-5-methyl-3-phenylisoxazole-4-carboxamide;

Compound C: N-{3-[4-[5-fluoro-2-(2,2,2-trifluoroethoxy)phenyl]-1-piperazinyl]propyl}-3-phenyl-5-methylisoxazole-4-carboxamide;

Compound D: 3-(2-chlorophenyl)-5-methyl-N-{3-[4-[2-(2,2,2-trifluoroethoxy)phenyl]-1-piperazinyl]propy}isoxazole-4-carboxamide; and

Compound E: N-{3-[4-[5-fluoro-2-(2,2,2-trifluoroethoxy)phenyl]-1-piperazinyl]propyl}-3-methyl-4-keto-2-phenyl-4H-1-benzopyran-8-carboxamide

Synthesis of compound A and related compounds of formula I are described in U.S. patent application Ser. Nos. 09/627,766 and 09/627,767.

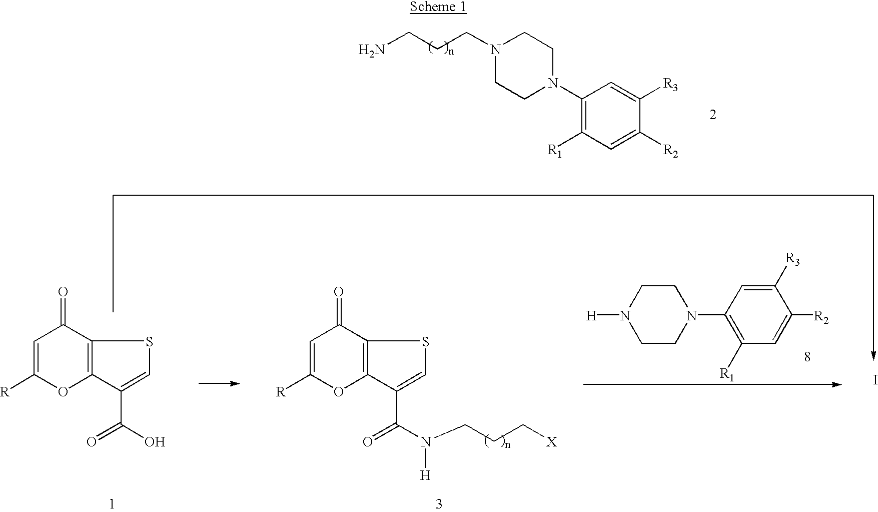

Direct condensation of 7-oxo-7H-thieno[3,2-b]pyran-3-carboxylic acids of the formula I with the ω-aminoalkylamino derivatives 2 (Scheme 1) leads to the compounds of the invention. The condensation can be carried out in the presence of a condensing agent (e.g., dicyclohexylcarbodiimide or diethyl cyanophosphonate) optionally in the presence of a promoting agent (e.g., N-hydroxysuccinimide, 4-dimethylaminopyridine or N,N′-carbonyldiimidazole) in an aprotic or chlorinated solvent (e.g., N,N-dimethylformamide or chloroform) at −10/140° C. (Albertson, 1962, Org. React., 12:205-218; Doherty et al., 1992, J Med. Chem., 35:2-14; Ishihara, 1991, Chem. Pharm. Bull., 39:3236-3243). In some cases the activated ester or amide intermediates (such as N-hydroxysuccinimide esters or acyl imidazolides) can be isolated and further reacted with 2 to be transformed into the corresponding amides (I) in an aprotic or chlorinated solvent at 10/100° C.

Another activated intermediate which can be used is the mixed anhydride of 1, obtainable reacting 1 with an alkyl chloroformate in the presence of a tertiary amine (e.g., triethylamine or N-methylmorpholine), which is reacted with 2 at 0-80° C.; optionally a promoting agent (e.g., 1-hydroxypiperidine) may be added before the amine addition (Albertson, 1962, Org. React., 12:157).

Alternatively the condensation can be carried out without a solvent at 150-220° C. (Mitchell et al., 1931, J Am. Chem. Soc., 53:1879) or in high-boiling ethereal solvents (e.g., diglyme).

The condensation can also be performed through preparation and optional isolation of reactive derivatives of 1 such as acyl halides. Preparation and use of these derivatives are well documented in the literature and known to people skilled in the art.

Also less reactive derivatives of 1 can be used, such as alkyl esters, which in turn can be converted into I in the presence of a condensing agent (e.g., trimethylaluminum) in an aprotic and/or chlorinated solvent (e.g., hexane, dichloromethane) at −10/80° C., or without any solvent at 80-180° C., (Weinreb et al., 1977, Tetrahedron Lett., 4171; Lipton et al., 1979, Org. Synth., 59:49).

By the same methods of condensation reported above and using H₂NCH₂(CH₂)ₙCH₂X (with X=halogen or OH) as a reagent, 1 can be transformed into 3. In the case of X=OH, the alcoholic group is then converted into a suitable leaving group by methods well known to those skilled in the art. Compounds 3 (with X=leaving group such as halogen or alky/arylsulphonyloxy group) can be subsequently reacted with an appropriate phenylpiperazine 8. The nucleophilic substitution is carried out preferably, but not necessarily, at a temperature within the range of 20-200° C. in a polar solvent such as N,N- dimethylformamide, acetonitrile, methanol, or without any solvent, usually in the presence of a base such as potassium carbonate. See also Gibson's chapter in Patai, 1968: “The Chemistry of the Amino Group”, p. 45 et seq., Wiley International Science, New York.

The preparation of compounds 2 is disclosed in the literature and is well known to those skilled in the art, and includes nucleophilic substitution of a phenylpiperazine 8 on a N-(ω-haloalkyl)phthalimide or a proper ω-haloalkylnitrile or haloalkylamide by the method illustrated above for the condensation of compounds 3 and 8, or by addition of an α, β-unsaturated alkylnitrile or alkylamide in a proper solvent (e.g., acetonitrile, N,N-dimethylformamide, a chlorinated solvent or other aprotic polar solvent) at a temperature between 0° C. and the reflux temperature of the solvent. Standard phthalimido-group deprotection or reduction of the amido or cyano group then provides compounds 2, and can be performed by methods well known to those skilled in the art.

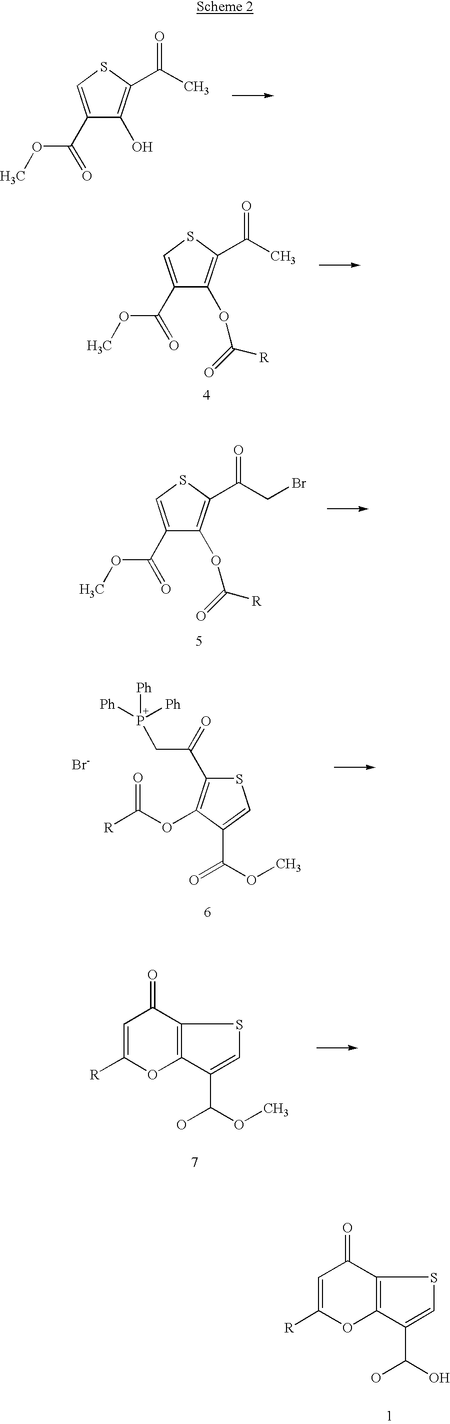

The acids 1 of the invention in which R represents cycloalkyl or phenyl group can be synthesized (Scheme 2) starting from methyl 2-acetyl-3-hydroxythiophene4-carboxylate (prepared as described in J. Chem. Soc. Perkin Trans I, 507, 1986), which can be esterified with the proper alkanoyl or aroyl chloride by using methods well known to those skilled in the art. Alternative procedures include the same methods described above for the amidification of 1, which could be applied as well in the esterification step to afford 4.

Monobromination of the methylketo group of 4 can afford 5, which can then be reacted with triphenylphosphine (typically by reflux in acetonitrile, toluene, or other aprotic solvent), to give the phosphonium salt 6. A subsequent intramolecular ester-Wittig reaction applied to this substrate yields the thieno[3,2-b]pyranes, 7. Hydrolysis of the ester group of compounds 7 by acid- or base-catalyzed procedures that are well known to those skilled in the art, yields compounds 1.

Well-known hydrolysis procedures include the use of sodium hydroxide in aqueous ethanol at 40-75° C., or lithium hydroxide in aqueous dimethylformamide, dioxane or tetrahydrofuran at 40-100° C.

The compounds 1 where R is a polyfluoroalkyl group can be prepared from 2-acetyl-3-hydroxythiophene-4-carboxylate following the cyclization procedure described by Riva et al., (1997, Synthesis, 195-201) by direct cyclization in the presence of anhydrous polyfluoroalkanoyl anhydrides catalysed by 1,8-diazabicycloundec-7-ene.

Compounds I where R₁ is a trifluoromethanesulphonyloxy group can be synthesized starting from compounds I where R₁ is a hydroxy group using procedures, which include the use of trifluoromethanesulphonic anhydride or N-phenyltrifluoromethane sulphonimide in aprotic solvents such as 1,2-dichloroethane or other chlorinated solvents or toluene, at a temperature in the range between 20° C. and the temperature of reflux of the solvent (Hendrickson et al., 1973, Tetrahedron Letters, 4607-4510). The N-oxides of the compounds I may be synthesized by simple oxidation procedures known to those skilled in the art. The oxidation procedure described in P. Brougham in Synthesis, 1015-1017 (1987) allows differentiation of the two nitrogen atoms of the piperazine ring and both the N-oxides and N,N′-dioxides to be obtained.

Preparation of the phenylpiperazines 8, which has not been described in the literature, is well documented in the experimental section and uses synthetic procedures well known to those skilled in the art, which comprise the synthesis of the proper aniline through standard reactions and the subsequent cyclization with bis-(2-chloroethyl)amine to afford the piperazine following the method of Prelog (1933, Collect. Czech. Chem. Comm., 5:497-502) or its variations (Elworthy, 1997, J. Med. Chem., 40:2674-2687).

Syntheses of compounds B, C, D and related compounds of Formula II are described in U.S. patent application Ser. No. 09/691,778. The general synthetic methods are described below.

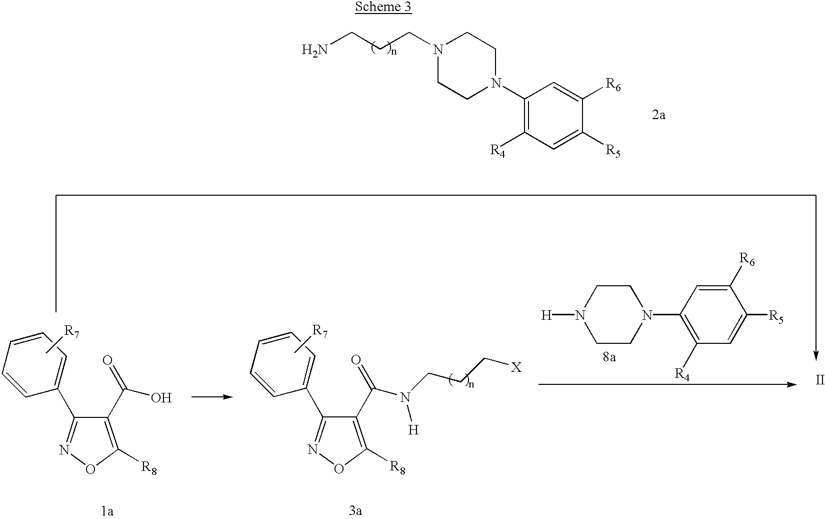

Direct condensation of compounds 1a, 3-arylisoxazole-4-carboxyl acids derivatives, with the ω-aminoalkyl derivatives 2a (Scheme 3) leads to the compounds of the invention. The condensation can be carried out in presence of a condensing agent (e.g., dicyclohexylcarbodiimide or diethyl cyanophosphonate) optionally in the presence of a promoting agent (e.g., N-hydroxysuccinimide, 4-dimethylaminopyridine or N,N′-carbonyldiimidazole) in an aprotic or chlorinated solvent (e.g., dimethylformamide or chloroform) at −10/140° C. (Albertson,1962, Org. React., 12:205-218; Doherty et al., 1992, J Med. Chem., 35:2-14; Ishihara et al., 1991, Chem. Pharm. Bull., 39:3236-3243). In some cases the activated ester or amide inter mediates (such as O-(N-succinimidyl)esters or acyl imidazolides) can be isolated and further reacted with 2a to be transformed into the corresponding amides (II) in an aprotic or chlorinated solvent at 10/100° C. Another activated intermediate which can be used is the mixed anhydride of 1a, obtainable by reacting 1a with an alkyl chloroformate in presence of a tertiary amine (e.g., triethylamine or N-methylmorpholine), then reacted with 2a at 0-80° C. Optionally a promoting agent (e.g., 1-hydroxypiperidine) may be added before the amine addition (Albertson,1962, Org. React., 12:157).

Alternatively, the condensation can be carried out without any solvent at 150-220° C. (Mitchell et al., 1931, J. Am. Chem. Soc., 53:1879) or in high-boiling ethereal solvents (e.g., diglyme).

The condensation can also be performed through preparation and optional isolation of reactive derivatives of 1a, such as acyl halides. Preparation and use of these derivatives are well documented in the literature and known to people skilled in the art.

Also less reactive derivatives of 1a can be used, such as alkyl esters, which, in turn, can be converted into II in the presence of a condensing agent (e.g., trimethylaluminum) in an aprotic and/or a chlorinated solvent (e.g., hexane, dichloromethane) at −10/80° C., or without any solvent at 80-180° C., (Weinreb et al., 1977, Tetrahedron Lett., 4171; Lipton et al, 1979, Org. Synth., 59:49).

By the same methods of condensation reported above and using H₂NCH₂(CH₂)ₙCH₂X (with X=halogen or OH) as a reagent, 1a can be transformed into 3a. In the case of X=OH, the alcoholic group is then converted into a suitable leaving group by methods well known to those skilled in the art. Compounds 3a (with X=leaving group such as halogen or aryl/alkylsulphonyloxy group) can be subsequently reacted with an appropriate phenylpiperazine 8a. The nucleophilic substitution is carried out preferably, but not necessarily at a temperature within the range of 20-200° C. in a polar solvent such as N, N-dimethyl formamide, acetonitrile or methanol, or without any solvent, usually in the presence of a base such as potassium carbonate. See also Gibson's chapter in Patai, 1968: “The Chemistry of the Amino Group”, p. 45, Wiley Int. Sci., New York.

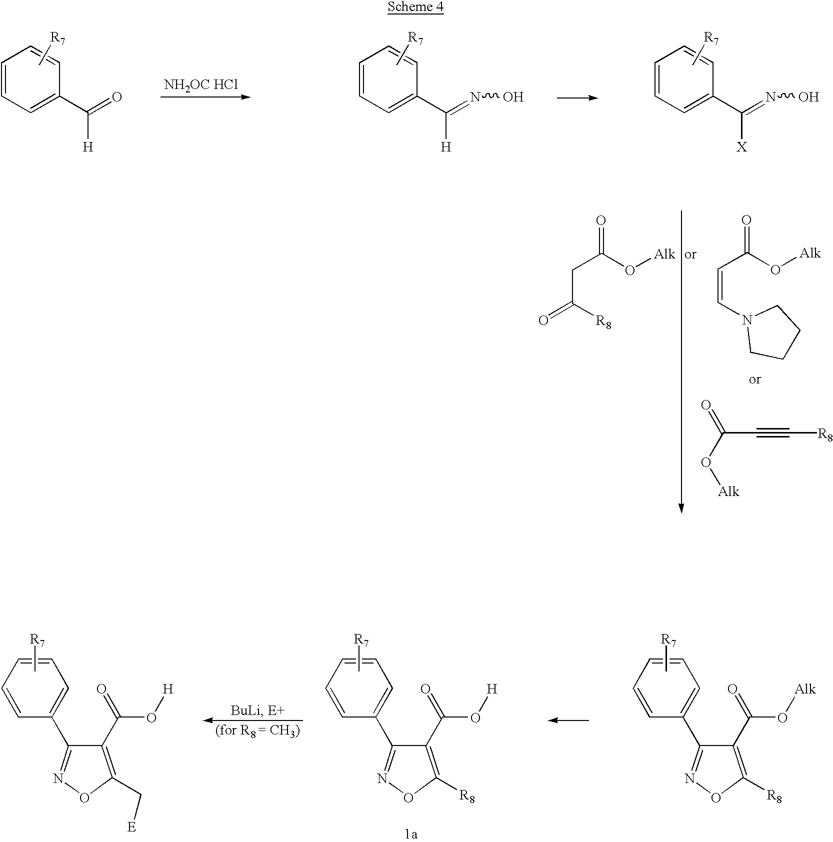

The preparation of compounds 1a (Scheme 4), which are not commercially available, is disclosed in detail in the literature and is well known to those skilled in the art and is usually carried out performing 1,3-dipolar cycloaddition reactions on benzohydroxamoyl halides (usually prepared by halogenation reaction on properly substituted benzaldoximes with alkaline halides or hypohalides or N-chloro-(or bromo)succinimide) with β-ketoesters or alkyl β-aminoacrylates (R₈═H) or alkyl propiolates in alkaline condition in a proper solvent (e.g., N,N-dimethylformamide, ethanol, diethyl ether, chlorinated solvents at a temperature in the range between −20° C. and solvent reflux, usually carrying out the reactions at 20-30° C.). (Scheme 4) Also see J Chem. Soc. 1963, 5838-5845 and 5845-5854; J Am. Chem. Soc. 1985, 107, 2721-2730, J Agric. Food Chem. 1995, 43, 219-228; U.S. Pat. No. 4,144,047.

Variations of the substitution at position R₈ can be obtained by using properly substituted β-ketoesters or alkyl propiolates or by reacting the lithium carbanion of the methyl derivatives 1a (R₈═CH₃) with various electrophiles in aprotic solvents such as tetrahydrofaran, diethyl ether, benzene, toluene or others at a temperature between −78° C. and the reflux temperature of the solvent (J. Org. Chem. 1985, 50, 5660-5666; J Med. Chem. 1988, 31, 473-476; J Med. Chem. 1990, 33, 2255-2259). The carboxylic functionality can be protected or not protected.

Compounds II where R₄ is a trifluoromethanesulphonyloxy group can be synthesised starting from compounds II where R₄ is a hydroxy group by known procedures that include the use of trifluoromethanesulphonic anhydride or N-phenyltrifluoromethanesulphonimide in aprotic solvents such as, for example, 1,2-dichloroethane or other chlorinated solvents, toluene, at a temperature in the range between −20° C. and the reflux temperature of the solvent (Hendrickson et al., 1973, Tetrahedron Letters, 4607-4610).

The N-oxides of compounds II may be synthesised by simple oxidation procedures known to those skilled in the art. The oxidation procedure described by Brougham (1987, Synthesis, 1015-1017) allows differentiation of the two nitrogen atoms of the piperazine ring, permitting both the N-oxides and the N, N′-dioxide to be obtained.

Synthesis of compound E and related compounds of Formula III is disclosed in U.S. patent application Ser. No. 09/691,770. The general synthetic methods are described below.

The condensation of acids 1b with ω-aminoalkylamino derivatives 2b (Scheme 5) can be carried out in the presence or absence of a condensing agent (e.g. dicyclohexylcarbodiimide or diethyl cyanophosphonate) optionally in the presence of a promoting agent (e.g. N-hydroxysuccinimide, 4-dimethylaminopyridine or N,N′-carbonyldiimidazole) in a polar aprotic or chlorinated solvent (e.g., dimethylformamide or chloroform) at −10/140° C. (Albertson N. F., 1962, Org. React. 12: 205-218; Doherty et al., 1992, J. Med. Chem., 35: 2-14; Ishihara et al., 1991, Chem. Pharm. Bull., 39: 3236-3243).

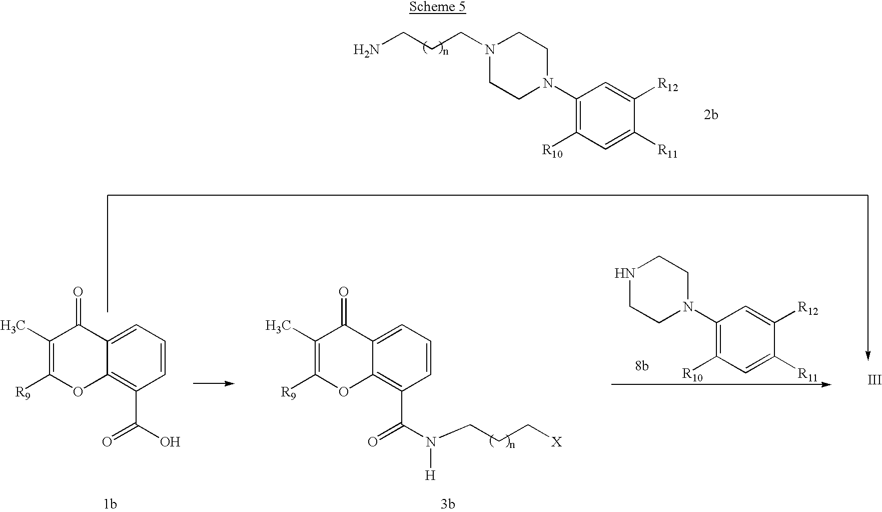

In some cases the intermediate esters or amides (such as N-hydroxysuccinimidyl esters or acyl imidazolides) can be isolated and further reacted with 2b to be transformed into the corresponding amides (III) in polar aprotic or chlorinated solvent at 10/100° C. Another intermediate which can be used is the mixed anhydride obtainable by reacting 1b with an alkyl chloroformate in the presence of a tertiary amine (e.g., triethylamine or N-methylmorpholine) followed by addition of 2b at 0-80° C., optionally a promoting agent (e.g., 1-hydroxypiperidine) may be added before the amine addition (Albertson N. F., 1962, Org. React. 12:157).

Alternatively the condensation can be carried out without solvent at 150-220° C. (Mitchell et al., 1931, J. Am. Chem. Soc. 53:1879) or in high-boiling ethereal solvents (e.g., diglyme). The condensation can also be performed through isolation of reactive derivatives of 1b such as acyl halides. Preparation and use of these derivatives are well documented in the literature and known to people skilled in the art.

Also less reactive derivatives of 1b can be used, such as alkyl esters, which, in turn, can be converted into III in the presence of a condensing agent (e.g., trimethylaluminium) in an aprotic and/or chlorinated solvent (e.g., hexane, dichloromethane) at −10/80° C., or without any solvent at 80-180° C. (Weinreb et al., 1977, Tetrahedron Lett. 48:4171; Lipton et al., 1979, Org. Synth. 59:49).

By the same methods of condensation reported above, using H₂NCH₂(CH₂)ₙCH₂X (with X=halogen or OH) as a reagent, derivatives 1b can be converted into the corresponding derivatives 3b. Compounds 3b ( with X=halogen or a leaving group), can be subsequently reacted with the appropriate phenylpiperazine 8b directly or by two sequential reactions, in the case of X=OH derivatives, which include conversion of the alcoholic group into a suitable leaving group by methods well known to those skilled in the art. The nucleophilic substitution on 3b to give III is preferably, but not necessarily, carried out at a temperature within the range of 20-160° C. in a polar solvent such as dimethylformamide, acetonitrile, methanol, or without any solvent, in the presence of a base such as potassium carbonate. See also Gibson's chapter in Patai, 1968 The Chemistry of the Amino Group, p. 45, Wiley Int. Sci., New York.

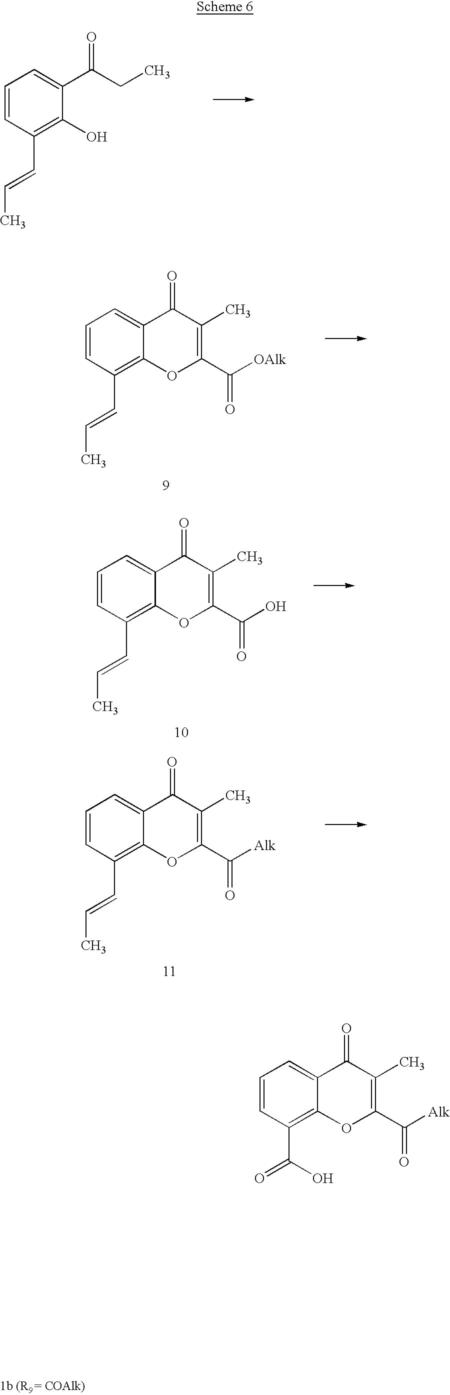

The compounds 1b of the invention in which R₉ represents an alkylcarbonyl group (Scheme 6) can be synthesized starting from 2-hydroxy-3-(1-propenyl)propiophenone which is condensed with excess diethyl oxalate in the presence of a base (e.g., sodium ethoxide, sodium hydride, sodium metal, lithium or sodium amide, potassium t-butoxide, lithium hexamethyldisilyl azide) in a suitable solvent such as ethanol, toluene, dioxane, tetrahydrofuran, 1,2-dichlorobenzene (or other aprotic solvent) or without any solvent at a temperature in the range between 20° C. and the reflux temperature of the reaction mixture (March J., 1992, Advanced Organic Chemistry, J. Wiley, Part 2 Chapter 10, 491-493; Schmutz, J., 1951, Helvetica Chimica Acta, 767-779). The intermediate crude αγ-diketoester is directly cyclized to give 9 (Alk=C₁₋₄ alkyl), with no purification, by acid catalysis (e.g., 37% HCl, 98% H₂SO₄, glacial acetic acid, trifluoroacetic acid, perchloric acid) in an appropriate solvent such as ethanol, toluene, a chlorinated solvent, or without any solvent, at a temperature in the range between 20° C. and the reflux temperature of the reaction mixture (Bryan J. D., 1960, J Chem. Soc Perkin Trans. 1:1279-1281).

Hydrolysis of the ester function of 9, by acid or base catalysis using methods well known to those skilled in the art, affords Compounds 10. Well known procedures include the use of sodium hydroxide in aqueous ethanol at 40-75° C. or lithium hydroxide in aqueous dimethylformamide or dioxane or tetrahydrofuran at 40-100° C.

Compounds 10 can be converted into keto derivatives 11 by direct reaction of lithium carboxylate with alkyl lithium derivatives (Rubottom G. M., 1983, J Org. Chem. 48:1550-1552). Alternatively, by conversion of the carboxy group into a more reactive C(O)X group, where X is 1-imidazolyl, chloro or bromo, OC(O)R or other reactive group, and then continuing the reaction with, for example, Meldrum's acid to afford an enolacyl derivative that can be hydrolyzed with acetic acid to give 11 or, alternatively, with the magnesium salt of a suitable β-diester (such as di-t-butyl malonate or diethyl malonate) to afford the corresponding β-ketoester to be hydrolyzed to 11.

Subsequent oxidative cleavage of the exocyclic double bond by permanganate oxidation (or other oxidative method well known to those skilled in the art; see, for example, Haines A. H., 1985, Methods for the oxidation of organic compounds, Academic Press, Chapter 3, part 5, 146-151) yields the desired carboxylic acids 1b having R₉═C(O)Alk

Acids 1b with R₉ is a COOAlk group can be clearly prepared from intermediates 9 carrying out the double-bond oxidation step as described above for 11.

Acids 1b in which R₉ is a CONR₁R₂ group can be prepared from intermediates 10 through an amidification reaction, which is well known to those skilled in the art, such as that described for 1b, with ammonia or an appropriate amine, then carrying out the double-bond oxidation step as described above. Due to the mild conditions (EP 0625522, Sohda et al.), a preferred method of amidification includes conversion of 10 to the respective acyl chloride by the use of oxalyl chloride.

Compounds III in which R₉ is a cyano group be obtained from compounds III with R₉═CONH₂ by a dehydration reaction through the use of triphenylphosphine in carbon tetrachloride or toluene or other suitable solvent at room temperature- reflux or, preferably, by the use of phosphorous-oxychloride/dimethylformamide or by other dehydration methods known to those skilled in the art (March J., 1992, Advanced Organic Chemistry, J. Wiley, Part 2, Chapter 7, part 39, 1041-1042).

Compounds III in which R₉ is a NHCOOAlk group can be prepared from intermediates 10 by Curtius rearrangement (March J., 1992, Advanced Organic Chemistry, 4^{th} edition, J. Wiley, ed., pages 1091-1092) carried out with diphenylphosphoryl azide and triethylamine in an appropriate alkanol at reflux or in a mixture of acetonitrile (or other solvent) and the appropriate alkanol. Oxidation of these intermediates as above affords acids 1b with R₉═NHCOOAlk.

Compounds III in which R₁₀ is a trifluoromethanesulphonyloxy group can be synthesized starting from compounds III in which R₁₀ is a hydroxy group by well-known procedures that include the use of trifluoromethanesulphonic anhydride or N-phenyltrifluoromethanesulphonimide in aprotic solvents such as 1,2-dichloroethane or other chlorinated solvents or toluene at a temperature in the range between −20° C. and the reflux temperature of the solvent (Hendrickson J. B., 1973, Tetrahedron Letters, 46:4607-4610).

The N-oxides of compounds III can be synthesized by simple oxidation procedures well known to those skilled in the art. The oxidation procedure described by P. Brougham et al., 1987, Synthesis, 1015-1017), allows the two nitrogen of the piperazine ring to be differentiated, allowing both the N-oxides and N,N′-dioxide to be obtained.

The compounds belonging to the class of α₁-receptor antagonists are well known. Screening α₁ antagonists to identify candidate compounds that are useful in practicing the present invention involves:

1) evaluating their affinity and selectivity in binding α₁ₐ , α_{1b} and α_{1d} subtypes of α₁-adrenergic receptor; and

2) confirming their pharmacological activity using one or more animal model of lower-urinary-tract dysfunction.

Affinity of the compounds of the invention for each subtype of the α₁-receptor can be assessed by receptor binding assays using, for example, the specific ligand ³H-prazosin, according to Testa et al., 1995, Pharmacol. Comm. 6:79-86).

Other assays that may also be used to measure binding of α₁-antagonists to α₁-subtypes are also encompassed by the present invention.

The binding affinity of a molecule can be measured for different subtypes of the α₁-adrenergic receptors, and the concentration at which a test compound inhibits binding of a control compound (e.g., prazosin) to a given receptor can be calculated using regression analysis, or equivalent computational methods that are well-known in the art (Tallarida et al., 1981, Manual of Pharmacologic Calculations. Springer-Verlag, pp. 10-12). These results are typically expressed as Ki. The results from these assays are used to calculate a measure of receptor selectivity, expressed as the ratio of affinities (Ki) for a given pair of receptors.

As discussed above, compounds useful in practicing the present invention bind selectively to α₁ₐ and α_{1d} receptors relative to the α_{1b} receptor. It will be understood that measurements of the relative affinity of a particular compound may vary depending upon the source of the receptor, as well as specific assay conditions.

A compound is considered to be a “selective” for α₁ₐ and α_{1d} receptors relative to the α_{1b} receptor if it exhibits a selectivity ratio of at least 10-fold for α₁ₐ versus α_{1b} (i.e., the Ki for α₁ₐ subtype is at least 10-fold below the Ki for α_{1b} subtype) and at least 6-fold for α_{1d} receptor versus α_{1b} (i.e., the Ki for α_{1d} subtype is at least 6-fold below the Ki for α_{1b} subtype). Additionally, the selectivity ratio of α₁ₐ versus α_{1d} subtypes should be lower than 10.

When a compound is found to be selective for α₁ₐ and α_{1d} subtypes versus α_{1b} subtype, its pharmacological activity can be confirmed using one or more animal model systems for dysfunction of the lower urinary tract.

A useful animal model system for measuring such pharmacological activity is, without limitation, cystometry in conscious rats with partial bladder-neck obstruction. This model measures detrusor contractions during filling which do not cause urine expulsion (unstable-bladder contractions). This model is reported in the literature as related to LUTS occurring in patients having obstructive urethral syndromes (Michel, 2000, Drugs of Today. 386 (Supp. B2): 3-6)

### Therapeutic Applications

The invention encompasses pharmaceutical formulations comprising those listed above, as well as methods employing these formulations for treating dysfunction of the lower urinary tract such as dysuria, incontinence, and enuresis. Dysuria includes urinary frequency, nocturia, urgency, and difficulty in emptying the bladder, i.e., a suboptimal volume of urine is expelled during micturition. Incontinence syndromes include stress incontinence, urgency incontinence, and overflow incontinence. Enuresis refers to the involuntary passage of urine at night or during sleep.

An “effective” amount of the compound for treating a urinary disorder is an amount that results in measurable amelioration of at least one symptom or parameter of the disorders described above.

An effective amount for treating the disorder can easily be determined by empirical methods known to those of ordinary skill in the art, such as by establishing a matrix of dosages and frequencies of administration and comparing a group of experimental units or subjects to each point in the matrix. The exact amount to be administered to a patient will vary depending on the state and severity of the disorder and the physical condition of the patient. A measurable amelioration of any symptom or parameter can be determined by a physician skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician or other practitioner.

For example, a single patient may suffer from several symptoms of dysuria simultaneously, such as, for example, urgency and frequency, either or both of which may be reduced using the methods of the present invention. In the case of incontinence, any reduction in the frequency or volume of unwanted passage of urine is considered a beneficial effect of the present methods of treatment, and thus, an amelioration of a symptom.

The guidelines given below are may be used for effective oral, parenteral and intravenous doses expressed as mg/kg of body weight daily, to be used in obstructive symptoms of the lower urinary tract:

| | |
|---|---|
| general | 0.001 to 20 |
| preferred | 0.05 to 3 |
| much preferred | 0.5 to 2 |

The much preferred values refer to oral administration. Intravenous doses should be 10 to 100 times lower. Doses for selective use, i.e., doses which are active in the lower urinary tract with no substantial effect on blood pressure, depend upon the particular compound used. Usually, in the case of compounds which selectively inhibit urethral contraction, up to four times the ED₅₀ amount used to inhibit urethral contractions can be administered with no substantial effect on blood pressure. Further dose refinement and optimization is possible simply using routine experiments. The active compounds of the invention can be administered orally, for example with an inert diluent or edible vehicle, or can be enclosed in gelatine capsules, or can be compressed into tablets. For oral therapeutic administration, the active compounds of the invention can be incorporated into excipients and used as tablets, troches, capsules, elixirs, suspensions, syrups, wafers, chewing gums and the like. These preparations should contain at least 0.5% of active compound, but the amount of active ingredient may vary depending upon the particular form and can conveniently vary from 5% to about 70% of the weight of the unit. The amount of active ingredient in these compositions is such as to allow an exact dosage to be obtained even when the desired dosage can be obtained by administering a plurality of dosage forms. The preferred compositions and preparations of the invention are prepared in such a manner that an oral dosage unit contains 0.1 to 300 milligrams of active compound. Tablets, pills, capsules, troches and the like can further contain, for example, the following ingredients: a ligand such as microcrystalline cellulose, tragacanth and gelatine; an excipient such as starch or lactose; a disintegrating agent such as alginic acid, sodium starch glycolate, maize starch and the like; a lubricant such as magnesium stearate and hydrogenated castor oil; a gliding agent such as colloidal silica; and a sweetener such as sucrose or saccharin or a flavour such as peppermint, methyl salicylate or orange flavour can be added. When the dosage unit form is a capsule, this can contain a fluid vehicle such as a fatty oil in addition to the above materials. Other dosage unit forms may contain various other materials which modify the physical form of the unit, e.g. coatings. Therefore, tablets and pills can be coated with sugar, shellac or other agents for enteric coating. A syrup may contain, in addition to active compounds, sucrose as a sweetener and certain preservatives, dyes and flavours. The materials used in the preparation of these various compositions should be pharmaceutically pure and nontoxic in the amounts used. For parenteral therapeutic administration, the active compounds of the invention can be incorporated into a solution or suspension. These preparations should contain at least 0.1% of active compound, but this may vary from 0.5 to about 30% of the weight of the preparations. The amount of active compound in these compositions is such as to allow an exact dosage to be obtained. Preferred compositions and preparations according to the present invention are prepared so that a parenteral unit dosage contains 0.2 to 100 milligrams of active compound. Solutions and suspensions can also contain the following ingredients: a sterile diluent such as water for injection, saline, fixed oils, polyethylene glycol, glycerine, propylene glycol and other synthetic solvents; antibacterial agents such as benzyl alcohol and methylparabens, antioxidants such as ascorbic acid and sodium disulphite, kelating agents such as ethylenediaminotetraacetic acid; buffers such as acetates; citrates and phosphates and agents for controlling tonicity such as sodium chloride and dextrose. Bottles for multiple parenteral doses can be of glass or plastic material. Other compositions suitable for administration by diverse routes of administration and containing compounds according to the present invention are also within the scope of the invention.

The dosage forms, further ingredients and routes of administration herein envisaged include those described in U.S. Pat. Nos. 4,089,969 and 5,091,182, all incorporated by reference in their entirety.

### EXAMPLE 1

### Compound A

### N-{3-[4-(2-Methoxyphenyl-1-piperazinyl]propyl}-7-keto-5-trifluoromethyl-7H-thieno[3,2-b]pyran-3-carboxamide

a) Methyl 7-keto-5-trifluoromethyl-7H-thieno[3,2-b]pyran-3-carboxylate (Compound 1A)

3.95 ml of trifluoroacetic anhydride and 9.2 ml of 1,8-diazabicyclo[5.4.0]undec-7-ene (DBU) were added at 0-5° C. to a mixture of 4.10 g of methyl 2-acetyl-3-hydroxy-thiophene-4-carboxylate (prepared as described in J. Chem. Soc. Perkins Trans I, 1986, 507) and 14 ml of pyridine. The mixture was heated at 80° C. for 27 hours. During this time three further additions of trifluoroacetic anhydride (9.9 ml in total) and DBU (9.2 ml) were made. After cooling to 20-25° C. the mixture was poured into ice (250 g) and 37% hydrochloric acid (50 ml) and extracted with ethyl acetate (2×80 ml). The combined organic layers were washed with water, dried over sodium sulphate and evaporated to dryness in vacuo. The residue was treated with petroleum ether-ethyl acetate 7:3 and filtered, and the filtrate purified by flash chromatography (petroleum ether-ethyl acetate, gradient from 7:3 to 0:1). The residue was dissolved in diethyl ether, washed with 5% aqueous sodium carbonate and then with water, dried over sodium sulphate and evaporated to dryness in vacuo to give the title compound (22%), melting at 148-158° C., which can be used in the next step without any further purification. The test sample was obtained by crystallisation from ethanol. M.p. 163-163° C.

| | |
|---|---|
| | Solvent: CDCl₃, |
| ¹H-NMR (200 MHz) spectrum | Chemical shift (δ) |

| | | | |
|---|---|---|---|
| 8.58 | s | 1H | H2 |
| 6.80 | s | 1H | H6 |
| 3.96 | s | 3H | COOCH₃ |

b) 7-keto-5-trifluoromethyl-7H-thieno[3,2-b]pyran-3-carboxylic acid (Compound 1B)

A mixture of 0.70 g of compound 1A, 5.6 ml of dioxane and 8.4 ml of 9N hydrochloric acid was refluxed for 75 minutes. After cooling to 20-25° C., the precipitated solid was filtered, washed with dioxane-water 1: 1.5 and then with water to give 0.46 g of the title compound as a grey solid, melting at 249-251 ° C.

| | |
|---|---|
| | Solvent: DMSO-d₆, |
| ¹H-NMR (200 MHz) spectrum | Chemical shift (δ) |

| | | | |
|---|---|---|---|
| 13.50 | bs | 1H | COOH |
| 8.25 | s | 1H | H2 |
| 7.19 | s | 1H | H6 |

c) N-{3-[4-(2-Methoxyphenyl)-1-piperazinyl]propyl}-7-keto-5-trifluoromethyl-7H -thieno[3,2-b]pyran-3-carboxamide

0.56 ml of 93% diethyl cyanophosphonate and 0.48 ml of triethylamine were added at 0° C. to a stirred solution of 0.82 g of compound 1B and 0.86 g of 1-(3-aminopropyl)-4-(2-methoxyphenyl)piperazine (prepared as described in patent GB 2,161,807) in 16 ml of anhydrous N,N-dimethylformamide. After 2 hours' stirring at 20-25° C. and 3 days' rest at the same temperature, the reaction mixture was poured into 150 ml of water and extracted with ethyl acetate. The organic layer was washed with water, dried over sodium sulphate and evaporated to dryness in vacuo. The crude was purified by flash chromatography (ethyl acetate-2.7N ammonia in methanol 95:5) to give the title compound as a light-brown solid, melting at 170-177° C. (33%).

| | |
|---|---|
| ¹H-NMR | |
| (200 MHz) spectrum | Solvent: CDCl₃, Chemical shift (δ) |

| | | | |
|---|---|---|---|
| 8.55 | s | 1H | H2 |
| 7.10 | t | 1H | NH |
| 6.85-7.10 | m | 4H | methoxyphenyl CHs |
| 6.80 | s | 1H | H6 |
| 3.88 | s | 3H | OCH₃ |
| 3.60 | 1 | 2H | NHCH₂ |
| 2.90-3.15 | m | 4H | 2 piperazine CH₂s |
| 2.45-2.80 | m | 6H | 2 piperazine CH₂s, CH₂CH₂CH₂N |
| 1.88 | dt | 2H | CH₂CH₂CH₂ |

### EXAMPLE 2

### Compound B

### N-{3-[4-(5-Chloro-2-methoxyphenyl)-1-piperazinyl]propyl}-5-methyl-3-phenylisoxazole-4-carboxamide

a) 1-(5-Chloro-2-methoxyphenyl)-4-[3-(N-phthalimido)propyl]piperazine (Compound 2A)

A mixture of 28.64 g of 1-(5-chloro-2-methoxyphenyl)piperazine, 44.6 g of anhydrous potassium carbonate and 33.65 g of N-(3-bromopropyl)phthalimide in 250 mL of acetonitrile was stirred at reflux for 8 hours. After cooling to room temperature, 800 mL of water was added under stirring and the resulting suspension was filtered by suction yielding a yellowish solid, which was washed with 300 mL of water and crystallized from methanol affording 46.5 g (91%) of the title compound, melting at 131-133° C.

| | |
|---|---|
| ¹H-NMR | |
| (200 MHz) spectrum; | Solvent: CDCl₃; Chemical shift (δ) |

| | | | |
|---|---|---|---|
| 7.78-7.82 | m | 2H | phthalimide H3, H6 |
| 7.64-7.78 | m | 2H | phthalimide H4, H5 |
| 6.92 | dd | 1H | methoxyphenyl H4 |
| 6.65-6.78 | m | 2H | methoxyphenyl H3, H6 |
| 3.81 | s | 3H | CH₃O |
| 3.71-3.89 | m | 2H | CH₂N(CO)₂ |
| 2.78-3.00 | m | 4H | 2 piperazine CH₂s |
| 2.40-2.65 | m | 6H | 2 piperazine CH₂s, CH₂CH₂CH₂N(CO)₂ |
| 1.80-2.03 | m | 2H | CH₂CH₂CH₂ |

b) 1-(3-Aminopropyl)-4-(5-chloro-2-methoxyphenyl)piperazine trihydrochloride. 2.15 H₂O (Compound 2B)

A solution of 20.7 g of Compound 2A and 8.6 mL of 85% hydrazine hydrate in 300 mL of 95% ethanol was stirred at reflux for 3.5 hours. Afterwards, the reaction mixture was cooled to room temperature, diluted with 400 mL of water, acidified with 37% hydrochloric acid (pH =1) and stirred for 0.5 hours. The precipitated solid was collected by filtration and washed with 1N hydrochloric acid followed by water. The filtrate was concentrated by evaporation in vacuo, filtered, made basic by the addition of 35% sodium hydroxide at 0-5° C. and extracted with diethyl ether. The organic layer was washed with brine, dried over sodium sulphate and evaporated to dryness in vacuo affording 13.6 g (96%) of the title compound as a base. Acidification of a solution of the base in chloroform with more than three equivalents of 3N ethanolic hydrogen chloride, followed by evaporation to dryness in vacuo and crystallisation of the residue from ethanol/diethyl ether 10:3, yielded the title compound, melting at 200-202° C.

| | |
|---|---|
| ¹H-NMR | |
| (200 MHz) spectrum; | Solvent: CDCl₃; Chemical shift (δ) |

| | | | |
|---|---|---|---|
| 11.20-11.50 | br | 1H | NH⁺ |
| 8.10-8.40 | br | 3H | NH3⁺ |
| 6.85-7.10 | m | 3H | phenyl H3, H4, H6 |
| 5.10 | br | 5.3H | NH⁺, 2.15 H₂O |
| 3.79 | s | 3H | CH₃O |
| 3.35-3.65 | m | 4H | 2 piperazine CH₂s |
| 3.03-3.35 | m | 6H | 2 piperazine CH₂s, CH₂CH₂CH₂NH₃⁺ |
| 2.80-3.03 | m | 2H | CH₂CH₂CH₂NH₃⁺ |
| 1.95-2.22 | m | 2H | CH₂CH₂CH₂NH₃⁺ |

c) N-{3-[4-(5-Chloro-2-methoxyphenyl)-1-piperazinyl]propyl}-5-methyl-3-phenylisoxazol -4-carboxamide

1.08 g of 93% diethyl cyanophosphonate and 0.92 mL of triethylamine were added to a mixture of 1.22 g of 3-phenyl-5-methylisoxazole-4-carboxylic acid (Aldrich), 1.87 g of Compound 2B as its base and 30 mL of anhydrous dimethylformamide stirred at 0-5° C. The temperature was allowed to rise to 20-25° C. and, after 3.5 hours' stirring, the mixture was poured into 300 mL of water and extracted with ethyl acetate. The combined organic layers were washed with 5% aqueous sodium carbonate and water. After drying on sodium sulphate, the solvent was removed in vacuo. The crude was crystallized from ethanol to yield 2.1 1 g (75%) of the title compound, melting at 139-142° C.

| | |
|---|---|
| | Solvent: CDCl₃; |
| ¹H-NMR (200 MHz) spectrum; | Chemical shift (δ) |

| | | | |
|---|---|---|---|
| 7.60-7.70 | m | 2H | phenyl H2, H6 |
| 7.45-7.55 | m | 3H | phenyl H3, H4, H5 |
| 6.95 | dd | 1H | methoxyphenyl H4 |
| 6.85 | d | 1H | methoxyphenyl H6 |
| 6.75 | d | 1H | methoxyphenyl H3 |
| 6.25 | t | 1H | NH |
| 3.82 | s | 3H | OCH₃ |
| 3.40 | q | 2H | NHCH₂ |
| 2.80-2.95 | m | 4H | 2 piperazine CH₂s |
| 2.69 | s | 3H | CH₃ |
| 2.40-2.55 | m | 4H | 2 piperazine CH₂s |
| 2.30 | t | 2H | CONHCH₂CH₂CH₂N |
| 1.55-1.70 | m | 2H | CH₂CH₂CH₂ |

### EXAMPLE 3

### Compound C

### N-{3-[4-[5-Fluoro-2-(2,2,2-trifluoroethoxy)phenyl]-1-piperazinyl]propyl}-3-phenyl-5-methylisoxazole-4-carboxamide

a) 5-Fluoro-2-(2,2,2-trifluoroethoxy)nitrobenzene (Compound 3A)

A stirred mixture of 3.14 g of 4-fluoro-2-nitrophenol, 13 g of cesium carbonate and 20 ml of anhydrous dimethylformamide was heated at 1 00° C. for 4 hours. 6.65 g of 2,2,2-trifluoroethyl p-toluenesulphonate was then added and the mixture was stirred at the same temperature for 40 hours. The solvent was then removed under reduced pressure at 35° C. and 50 ml of water was added to the residue. The mixture was acidified with 37% hydrochloric acid and extracted with 3×40 ml of ethyl acetate. The organic layer was washed with 20 ml of brine, dried over sodium sulphate and evaporated to dryness under reduced pressure. The residue was purified by flash chromatography (petroleum ether/ethyl acetate 100:7) to afford 1.53 g (32%) of Compound 3A as an oil.

| | |
|---|---|
| | Solvent: CDCl₃; |
| ¹H-NMR (200 MHz) spectrum; | Chemical shift (δ) |

| | | | |
|---|---|---|---|
| 7.65 | dd | 1H | H6 |
| 7.32 | ddd | 1H | H4 |
| 7.16 | dd | 1H | H3 |
| 4.42 | q | 2H | CH₂ |

b) 5-Fluoro-2-(2.2,2-trifluoroethoxy) aniline (Compound 3B)

A mixture of 0.66 g of Compound 3A and 0.07 g of Raney-Nickel in 20 mL of ethyl acetate was stirred for 14 hours at 20-25° C. The organic layer was separated and the mixture extracted with 2×40 mL of ethyl acetate. The combined organic layers were washed with 20 mL of brine, dried over sodium sulphate and evaporated to dryness in vacuo to afford 0.52 g (90.6%) of Compound 3B as an orange oil.

| | |
|---|---|
| | Solvent: CDCl₃; |
| ¹H-NMR (200 MHz) spectrum; | Chemical shift (δ) |

| | | | |
|---|---|---|---|
| 6.70 | dd | 1H | H6 |
| 6.28-6.50 | m | 2H | H3 and H4 |
| 4.32 | q | 2H | CH₂ |
| 3.92 | br | 2H | NH₂ |

c) 1-[5-Fluoro-2-(2,2,2-trifluoroethoxy)phenyl]piperazine (Compound 3C)

A stirred mixture of 0.52 g of Compound 3B, 0.45 g of bis-(2-chloroethyl)amine hydrochloride, 0.5 g of potassium iodide, 0.34 g of anhydrous potassium carbonate and 20 mL of n-butanol was refluxed for 32 hours under nitrogen. The solvent was removed under reduced pressure. The residue was treated with 10 mL of water and 10 mL of 20% aqueous sodium carbonate and extracted with 2×30 mL of ethyl acetate. The organic layer was washed with brine, dried over sodium sulphate and evaporated to dryness in vacuo. The residue was purified by flash chromatography (chloroform: 2N ammonia in methanol gradient from 100:3 to 100:5) to afford 0.1 g (14 %) of Compound 3C as an oil.

| | |
|---|---|
| | Solvent: CDCl₃; |
| ¹H-NMR (200 MHz) spectrum; | Chemical shift (δ) |

| | | | |
|---|---|---|---|
| 6.80-6.93 | m | 1H | H3 |
| 6.55-6.71 | m | 2H | H6, H4 |
| 4.36 | q | 2H | OCH₂CF₃ |
| 3.05 | br | 8H | piperazine CH₂s |
| 2.38 | s | 1H | NH |

d) N-(3-Chloropropyl)-3-phenyl-5-methylisoxazole-4-carboxamide (Compound 3D)

4.48 ml of 93% diethyl cyanophosphonate and 7.66 ml of triethylamine were added at 0° C. to a stirred mixture of 5.05 g of 3-phenyl-5-methylisoxazole-4-carbossylic acid, 3.57 g of 3-chloropropylamine hydrochloride and 50 ml of dimethylformamide. The temperature was allowed to rise to 20-25° C. and, after stirring for 3.5 hours, the mixture was poured into 100 ml of ice-cold water, the precipitated solid was filtered and washed on a funnel with a 2:1 mixture of water:dimethylformamide followed by water. Drying afforded the title compound (89%). M.p. 122-124° C.

| | | | |
|---|---|---|---|
| 7.45-7.60 | m | 5H | phenyl CHs |
| 5.50 | br | 1H | NH |
| 3.30-3.45 | m | 4H | CH₂CH₂CH₂ |
| 2.70 | s | 3H | CH3 |
| 1.80-1.90 | m | 2H | CH₂CH₂CH₂ |

e) N-{3-[4-[5-Fluoro-2-(2,2,2-trifluoroethoxy)phenyl]-1-piperazinyl ]propyl}-5-methyl-3-phenylisoxazole-4-carboxamide

A mixture of 0.3 g of Compound 3D, 0.29 g of Compound 3C and 0.14 g of anhydrous potassium carbonate was stirred at 160° C. for 20 minutes. After cooling to room temperature, the crude was purified by flash chromatography (dichloromethane/2N ammonia in methanol 97.5:2.5) to give the title compound (59%). M.p. 123-125° C.

¹H-NMR (200MHz) spectrum; Solvent: CDCl₃; Chemical shift (δ)

| | | | |
|---|---|---|---|
| 7.60-7.70 | m | 2H | phenyl H2, H6 |
| 7.45-7.55 | m | 3H | phenyl H3, H4, H5 |
| 6.80-6.90 | m | 1H | trifluoroethoxyphenyl H3 |
| 6.55-6.70 | m | 2H | trifluoroethoxyphenyl H4, H6 |
| 6.20 | t | 1H | NH |
| 4.30 | q | 2H | OCH₂CF₃ |
| 3.35 | q | 2H | NHCH₂ |
| 2.80-2.95 | m | 4H | 2 piperazine CH₂s |
| 2.65 | s | 3H | CH₃ |
| 2.35-2.45 | m | 4H | 2 piperazine CH₂s |
| 2.25 | t | 2H | CONHCH₂CH₂CH₂N |
| 1.50-1.65 | m | 2H | CH₂CH₂CH₂ |

### EXAMPLE 4

### Compound D

### 3-(2-Chlorophenyl)-5-methyl-N-{3-[4-[2-(2,2,2-trifluoroethoxy)phenyl]-1-piperaziny}propyl}-isoxazole-4-carboxamide

a) 1-[2-(2,2,2,-Trifluoroethoxy)phenyl]-4-[3-(N-phthalimido)propyl]piperazine (Compound 4A)

The title compound was prepared as in Example 2 for Compound 2A, replacing 1-[2-(2,2,2-trifluoroethoxy)phenyl]piperazine (prepared as described in patent EP 748800) for 1-(5-chloro-2-methoxyphenyl)piperazine. The reaction mixture was extracted with diethyl ether, the organic layer was dried over sodium phosphate and then filtered on a silica gel panel washing with diethyl ether. Evaporation to dryness in vacuo afforded the title compound (91%), melting at 111-113° C.

¹H-NMR (200MHz) spectrum; Solvent: CDCl₃; Chemical shift (δ)

| | | | |
|---|---|---|---|
| 7.60-7.92 | m | 4H | phthalimide CHs |
| 6.80-7.10 | m | 4H | trifluoroethoxyphenyl CHs |
| 4.35 | q | 2H | OCH₂CF₃ |
| 3.80 | t | 2H | (CO)₂NCH₂ |
| 2.75-3.12 | m | 4H | 2 piperazine CH₂s |
| 2.30-2.75 | m | 6H | (CO)₂NCH₂CH₂CH₂N, 2 piperazine CH₂s |
| 1.75-2.10 | m | 2H | CH₂CH₂CH₂ |

b) 1-(3-Aminopropyl)-4-[2-(2 2,2,-trifluoroethoxy)phenyl]piperazine (Compound 4B)

The title compound was prepared as described in Example 2 for Compound 2B, replacing Compound 4A for Compound 2A. Extraction of the alkalinised filtrate with dichloromethane, followed by purification by flash chromatography (ethyl acetate-2N ammonia in methanol 10:1) afforded the title compound as an oil (78%).

¹H-NMR (200MHz) spectrum; Solvent: CDCl₃; Chemical shift (δ)

| | | | |
|---|---|---|---|
| 6.82-7.12 | m | 4H | aromatics CHs |
| 4.4 | q | 2H | OCH₂CF₃ |
| 2.95-3.25 | m | 4H | 2 piperazine CH₂s |
| 2.72-2.85 | m | 2H | H₂NCH₂CH₂CH₂N |
| 2.52-2.72 | m | 4H | 2 piperazine CH₂s |
| 2.38-2.52 | m | 2H | H₂NCH₂CH₂CH₂N |
| 1.55-1.80 | m | 4H | H₂NCH₂CH₂CH₂N |

c) 3-(2-Chlorophenyl)-5-methyl-N-{3-[4-[2-(2,2,2-trifluoroethoxy)phenyl]-1-piperazinyl}propyl}-isoxazole-4-carboxamide

A mixture of 0.32 g of Compound 3B, 0.19 g of triethylamine, 0.31 g of 3-(2-chlorophenyl-5-methylisoxazole-4-carbonyl chloride (Lancaster) and 40 ml of dichloromethane was stirred at 20-25° C. for 24 hours. The solution was washed with 2N sodium hydrate (4 ×4 ml), dried over sodium sulphate and evaporated to dryness in vacuo. The crude was purified by flash chromatography (chloroform-2N ammonia in methanol 100:3) affording the title compound as an oil (67%).

¹H-NMR (200MHz) spectrum; Solvent: CDCl₃; Chemical shift (δ)

| | | | |
|---|---|---|---|
| 7.35-7.62 | m | 4H | chlorophenyl CHs |
| 6.82-7.12 | m | 4H | trifluoroethoxyphenyl CHs |
| 5.50-5.80 | br | 1H | NH |
| 4.40 | q | 2H | OCH₂CF₃ |
| 3.22-3.42 | m | 2H | NHCH₂ |
| 2.88-3.15 | m | 4H | 2 piperazine CH₂s |
| 2.78 | s | 3H | CH₃ |
| 2.35-2.63 | m | 4H | 2 piperazine CH₂s |
| 2.10-2.35 | m | 2H | CONHCH₂CH₂CH₂N |
| 1.45-1.75 | m | 2H | CH₂CH₂CH₂ |

### EXAMPLE 5

### Compound E

### N-{3-[4-[5-Fluoro-2-(2,2,2-trifluoroethoxy)phenyl]-1-piperazinyl]-propyl}-3-methyl-4-keto-2-phenyl-4H-1-benzopyran-8-carboxamide

The title compound was prepared following the procedure described in Example 3, using N-(3-chloropropyl)-3-methyl-4-keto-2-phenyl-4H-1-benzopyran-8-carboxamide (prepared as described by Leonardi et al. in U.S. Pat. No. 5,474,994), instead of Compound 3D and heating to 190° C. for 30 minutes. Purification was carried out by flash chromatography (chloroform/2N methanolic ammonia, gradient from 100:1 to 100:3) affording the title compound as an ivory solid (66.5%). M.p. 162-166° C.

¹H-NMR (200MHz, CDCl₃, δ)

| | | | |
|---|---|---|---|
| 8.38 | d | 2H | H5 and H7 |
| 7.75-7.80 | m | 2H | H2 and H6 of 2-phenyl ring |
| 7.55-7.75 | m | 4H | H3, H4 and H5 of 2-phenyl ring, CONH |
| 7.50 | t | 1H | H6 |
| 6.86 | dd | 1H | trifluoroethoxyphenyl H3 |
| 6.50-6.70 | m | 2H | trifluoroethoxyphenyl H4 and H6 |
| 4.31 | q | 2H | OCH₂CF₃ |
| 3.50-3.65 | m | 2H | CONHCH₂CH₂CH₂ |
| 2.85-3.05 | m | 4H | 2 piperazine CH₂s |
| 2.30-2.55 | m | 6H | 2 piperazine CH₂s, CONHCH₂CH₂CH₂ |
| 2.20 | s | 3H | CH₃ |
| 1.60-1.85 | m | 2H | CONHCH₂CH₂CH₂ |

### EXAMPLE 6

### Pharmacological Data

### Determination of Affinity for Cloned α₁-αdrenoceptor Subtypes (α₁ₐ, α_{1b}, α_{1d}) by Radioligand Binding Assay

Determination of affinity for cloned subtypes of α₁-adrenoceptor subtypes was performed in membranes from cells transfected by electroporation with DNA expressing the genes encoding each α₁-adrenoceptor subtype.

Cloning and stable expression of the genes expressing α₁-adrenoceptor subtypes were performed as previously described (Testa et al., 1995, Pharmacol. Comm. 6: 79-86, and cited references). The cell membranes were incubated in 50 mM Tris, pH 7.4, with 0.2 nM [³H]prazosin, in a final volume of 1.02 mL for 30 minutes at 25° C., in the absence or presence of competing drugs (1 pM-10 μM). Non-specific binding was determined in the presence of 10 μM phentolamine. Incubation was stopped by addition of ice-cold Tris buffer and rapid filtration through 0.2% polyethyleneimine-pretreated Schleicher & Schuell GF52 filters. Inhibition of specific binding of the radioligands by the test drugs was analyzed to estimate the IC₅₀ value by using the non-linear curve-fitting program Allfit (De Lean et al., 1978, Am. J Physiol. 235:E97-E102). The IC₅₀ value was converted to an affinity constant (Ki) by the equation of Cheng et al., 1973, Biochem. Pharmacol. 22:3099-3108. Data were expressed as mean Ki.

### Cystometry in Conscious Rats Obstructed by Partial Urethra Ligature

In order to obtain a partial obstruction of the urethra, the method previously reported by Malgren (Nalgren et al., 1987, J. Urol. 137:1291-1294; 1988, Neurourol. Urodyn. 6:371), was followed with minor modifications (Guarneri et al., 1991, Pharmacol. Res. 24:263).

Female rats of the Sprague-Dawley strain [Crl:CD(SD)BR, from Charles River Italia, Calco, Como, Italy] weighing 225-275 g were used. The animals were maintained in constant temperature and humidity conditions, on a forced 12-hour light-dark cycle and with food and water ad libitum for at least one week before the experiment.

After being anaesthetized with 3 ml/kg i.p. equitensin (pentobarbital 1.215 g, chloral hydrate 5.312 g, magnesium sulphate 2.657 g, ethanol 12.5 ml, propylene glycol 49.5 ml, distilled water to 125 ml of final volume), the rats were placed in a supine position and the bladder and urethra were exposed via an incision in the shaven abdomens and gently pulling away the muscle portion. The urethra was cannulated with a polyethylene tube with an outside diameter of 1.22 mm, and the urinary bladder was then emptied and, via the cannula introduced through the urethra, filled with physiological saline. A silk (Ethicon 3/0) ligature was placed around the urethra with the cannula inside and the intraurethral cannula was then removed. The abdominal incision was sutured and, immediately after the operative procedure, antibiotic medication (penicillin G 200 000 I.U./kg i.p. and streptomycin 260 mg/kg i.p.) was performed.

Three weeks after the procedure for partially obstructing the urethra, the animals were prepared for cystometry by surgical insertion into the bladder of a catheter, through which the bladder was gradually filled.

The rats, anaesthetised with equitensin 3 ml/kg i.p., were placed in a supine position and, via an incision of about 10 mm in the abdominal wall, the urinary bladder was exposed and gently freed from surrounding tissues.

The urinary bladder was emptied manually and cannulated, via a small incision at the bladder top, with a polyethylene cannula (type PE-50, 0.58 mm I.D. ×0.96 mm O.D.), which was permanently secured to the bladder with silk thread.

The cannula was exposed through a subcutaneous tunnel in the retroscapular area, where it was fastened with a plastic adapter, in order to avoid the risk of removal by the animal. After washing the urinary bladder with physiological saline, the catheter was sealed using a small flame and the abdominal incision was sutured.

To allow evaluation of the effect of a test compound after intravenous administration, the jugular vein was cannulated with a polyethylene cannula (type PE-50, 0.58 mm I.D.×0.96 mm O.D.) filled with heparinised physiological saline. As for the bladder catheter, this cannula, too, was exteriorised, secured and sealed in the retroscapular area.

Two days after the operation, the rats, fasted overnight, were placed in Bollman's cages or in Bollman's cages modified so as to have an opening in the bottom to allow collection of urinated fluid.

After an adaptation period of 20 minutes, the free end of the bladder cannula was connected to a pressure transducer and a special apparatus which allowed infusion into the urinary bladder of physiological saline at 37° C. at a constant rate of 10 ml/hour. Intravesical pressure changes caused by bladder filling were recorded by the pressure transducer which was connected to a recording polygraph.

From the cystometrograms, the frequency and mean amplitude of spontaneous bladder contractions not inducing micturition, termed “unstable-bladder contractions” (UBC), within 2 minutes prior to micturition were evaluated.

The frequency and amplitude of “UBC” were generally evaluated in one/two reproducible cystometrograms recorded before treatment and considered as baseline values.

After obtaining baseline cystometrograms, the compounds were administered.

The effect of the test compounds on ineffective emptying contractions was evaluated in the first, second and third cystometrograms after treatment. The highest percent change observed was considered to be a useable result.

Cystometry in conscious rats with partial urethra obstruction revealed detrusor contractions which were ineffective in urine expulsion (non-micturition contractions =unstable-bladder contractions (UBC)). This model is reported in the scientific literature as related to the lower-urinary-tract symptoms occurring in humans having obstructive urethral syndromes (Michel, 2000, Drugs of Today. 386 (Supp. B2):3-6).

The compounds used as comparisons for compounds A-D in binding and physiological studies included known compounds such as terazosin, prazosin and tamsulosin. Other compounds used in these studies include the following:

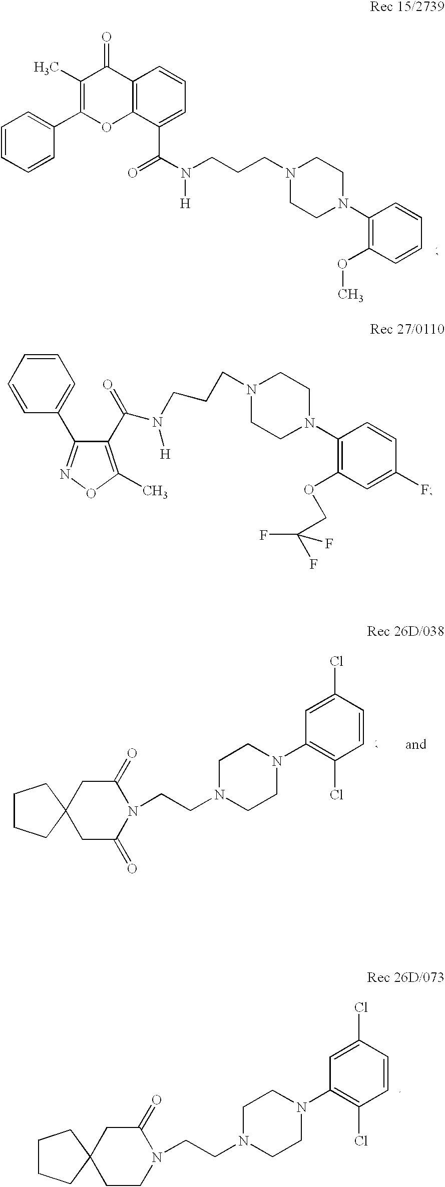

### Results

The results obtained with compounds having different receptor-affinity profiles are shown in Tables 1 and 2.

Generally, non-selective α₁-blockers (prazozin, terazosin) induced a marked and dose-dependent reduction in the number and amplitude of non-effective micturition contractions.

Tamsulosin, a compound partially selective for the α_{1d} adrenergic subtype, was found to be very potent. Its potency may be related to its higher affinity for this subtype.

The selective α₁ₐ -subtype agonists (Rec 15/2739, 27/0110), as well as the selective α_{1d}-subtype selective compound (Rec 26D/038, 26D/073) were poorly active as inhibitors of unstable-bladder contractions.

Compounds having selectivity for the α₁ₐ and α_{1d} subtypes versus the α_{1b} subtype (Compound B and Compound D), proved to be more potent than the molecules selective only for the α₁ₐ subtype or the α_{1d} subtype.

TABLE 1
Cystometry in conscious rats with partial urethral obstruction. Effects
on non-effective micturition contractions. Data represent the percent
inhibition of frequency and amplitude of non-effective contractions
observed for 2 minutes before micturition. Affinity of test compounds
for α-adrenoceptor subtypes is also shown in table.

| | | | | Obstructed Rat | | | | | | | |
|---|---|---|---|---|---|---|---|---|---|---|---|
| | Binding Affinity | | | % inhibition of | | | | % inhibition of | | | |
| | (Ki nM) | | | frequency mg/kg | | | | amplitude mg/kg | | | |
| Example | α₁ₐ | α_{1b} | α_{1d} | 0.1 | 0.3 | 1 | 3 | 0.1 | 0.3 | 1 | 3 |
| 15/2739 | 0.11 | 4.55 | 1.44 | | | 28 | | | | 29 | |
| 27/0110 | 0.35 | 69 | 18.9 | | | 27 | 49 | | | 21 | 53 |
| 26D/038 | 128 | 19 | 0.13 | | 38 | 27 | | | 39 | 21 | |
| 26D/073 | 42 | 11 | 0.11 | | 31 | 25 | | | 16 | 18 | |
| A | 0.6 | 23.16 | 3.6 | | 44 | 67 | | | | | |
| B | 0.8 | 23.6 | 2.7 | | | 49 | | | | 44 | |
| C | 0.05 | 11.52 | 0.33 | | | | | | | | |
| D | 0.07 | 14.1 | 0.49 | | | 68 | | | | 73 | |
| E | 0.07 | 3.54 | 0.32 | | | | | | | | |
| Terazosin | 6.9 | 2.2 | 2.4 | 30 | 65 | 93 | | 31 | 64 | 86 | |
| Prazosin | 0.61 | 0.42 | 0.23 | 48 | 74 | | | 63 | 87 | | |
| Tamsulosin | 0.06 | 0.94 | 0.16 | 61 | | | | 52 | | | |

TABLE 2
Cystometry in conscious rats with partial urethral obstruction. Effects on non-effective
micturition contractions. Data represent number (frequency) and amplitude (mmHg) of
non-effective micturition contractions observed for 2 minutes before micturition.

| | | | Frequency | | Amplitude | |
|---|---|---|---|---|---|---|
| Compound | Dose | n | Baseline | After Treat. | Baseline | After Treat. |
| Prazosin | 0.1 | 8 | 5.3 ± 0.4 | 2.8 ± 0.8** | 8.3 ± 1.1 | 3.1 ± 0.9** |
| | 0.3 | 7 | 2.3 ± 1.1** | 2.3 ± 1.1** | 15.9 ± 6.4 | 2.1 ± 1.5** |
| Terazosin | 0.1 | 5 | 5.7 ± 1.1 | 4.0 ± 0.3 | 9.5 ± 1.4 | 6.6 ± 0.7 |
| | 0.3 | 7 | 5.4 ± 0.5 | 1.9 ± 0.5** | 10.3 ± 1.9 | 3.7 ± 1.1** |
| | 1.0 | 4 | 3.8 ± 0.6 | 0.3 ± 0.3** | 5.3 ± 0.3 | 0.8 ± 0.8* |
| Tamsulosin | 0.1 | 8 | 4.9 ± 0.5 | 1.9 ± 0.7** | 7.7 ± 1.4 | 3.7 ± 1.1** |
| Rec 15/2739 | 1.0 | 6 | 6.7 ± 0.4 | 4.8 ± 0.2 | 10.2 ± 0.8 | 7.3 ± 1.3 |
| Rec 27/0110 | 1.0 | 7 | 6.1 ± 0.7 | 4.4 ± 0.8 | 7.6 ± 1.3 | 6.0 ± 1.4 |
| | 3.0 | 6 | 4.3 ± 0.5 | 2.2 ± 0.7 | 4.8 ± 0.4 | 2.3 ± 0.8 |
| Rec 26D/038 | 0.3 | 10 | 5.6 ± 0.5 | 3.6 ± 0.7** | 10.7 ± 1.3 | 6.6 ± 1.5** |
| | 1.0 | 4 | 6.1 ± 0.3 | 4.5 ± 1.2 | 9.4 ± 0.9 | 7.5 ± 1.6 |
| Rec 26D/073 | 0.3 | 8 | 4.0 ± 0.5 | 2.8 ± 0.7 | 7.4 ± 1.5 | 6.2 ± 1.6 |
| | 1.0 | 9 | 5.9 ± 0.5 | 4.4 ± 0.7 | 9.2 ± 1.6 | 7.5 ± 1.2 |
| B | 1.0 | 6 | 5.8 ± 0.4 | 3.0 ± 0.7** | 7.6 ± 0.7 | 4.2 ± 1.0** |
| D | 1.0 | 6 | 5.3 ± 0.7 | 1.7 ± 0.8** | 7.8 ± 2.0 | 2.1 ± 0.7** |

n (number of rats/group);
Dose (mg/kg)
p < 0.05;
**p < 0.01 versus basal values (before treatment).

### Example 7

### Effects of Test Compounds in Patients Suffering from Lower Urinary Tract Symptoms

Efficacy of compounds A, B, C, D and E in the treatment of lower-urinary-tract symptoms are tested in patients with these symptoms.

Compounds A-E are administered orally once or twice daily at doses of 5, 12.5, 25 and 100 mg for a period of 40 days. Total daily dosages, therefore, are 5, 10, 12.5, 25, 50, 100 or 200 mg.

The therapeutic effect of compounds A-E are measured by a questionnaire completed by the patients, which are used to determine, for example, micturition frequency, the number of micturition episodes during the night, the extent of difficult urination, the pain or feeling of discomfort in the lower abdominal tract or genital areas.

The efficacy of compounds A-E is measured on the basis of any amelioration observed for each symptom associated to lower-urinary-tract symptoms compared to a control group of patients who are administered placebo with the same administration method and regime.

We claim:

1. A method of treating lower-urinary-tract symptoms (LUTS) in a mammal in need of such treatment, comprising administering to said mammal a therapeutically effective amount of an α₁-adrenergic receptor ligand wherein said ligand binds to α₁ₐ-adrenergic receptor with an affinity at least about 10-fold greater than the affinity with which said ligand binds to the α_{1b}-adrenergic receptor, said ligand binds to α_{1d} adrenergic receptor with an affinity at least about 6-fold greater than the affinity with which said ligand binds to the α_{1b}-adrenergic receptor, and said ligand is not tamsulosin.

2. The method of claim 1 wherein the ligand binds to the α₁ₐ-adrenergic receptor with an affinity that is 1 to 10 times higher than the affinity with which the ligand binds to the α_{1d}-adrenergic receptor.

3. The method of claim 1 wherein the ligand binds to each of the α₁ₐ- and α_{1d}-adrenergic receptors with an affinity at least about 10-fold greater than said ligand binds to the α_{1b} adrenergic receptor.

4. The method of claim 3 wherein the ligand binds to each of the α₁ₐ- and α_{1d}-adrenergic receptors with an affinity at least about 20-fold greater than said compound binds to the α_{1b}-adrenergic receptor.

5. The method of claim 1 wherein said ligand is an α₁-adrenergic antagonist.

6. The method of claim 1 wherein said ligand has a Ki for the α₁ₐ-adrenergic receptor of from about 0.01 to about 100 nM.

7. The method of claim 6 wherein said ligand has a Ki for the α₁ₐ-adrenergic receptor of from about 0.05 to about 10 nM.

8. The method of claim 1 wherein said ligand has a Ki for the α_{1d}-adrenergic receptor of from about 0.01 to about 100 nM.

9. The method of claim 8 wherein said ligand has a Ki for the α_{1d}-adrenergic receptor of from about 0.1 to about 10 nM.

10. The method of claim 1 wherein said ligand has a Ki for each of the α₁ₐ- and α_{1d}-adrenergic receptors of from about 0.01 to about 100 nM.

11. The method of claim 10 wherein said ligand has a Ki for the α₁ₐ-adrenergic receptor of about 0.05 to about 10 nM and a Ki for the α_{1d}-adrenergic receptor of from about 0.1 to about 10 nM.

12. The method of claim 1 wherein the affinity of said ligand is greater for the α₁ₐ-adrenergic receptor than the α_{1d}-adrenergic receptor.

13. The method of claim 1 wherein the affinity of said ligand is greater for the α_{1d}-adrenergic receptor than the α₁ₐ-adrenergic receptor.

14. The method of claim 1 wherein said ligand has an affinity for the α_{1d}-adrenergic receptor that is within 10-fold of the affinity said ligand has for the α₁ₐ-adrenergic receptor.

15. The method of claim 1 wherein said ligand is administered via oral, transdermal, parenteral, intravenous, intramuscular, subcutaneous or transmucosal routes, or by inhalation.

16. The method of claim 1 wherein said ligand is administered as part of a pharmaceutically acceptable composition.

17. The method of claim 1 wherein said ligand is administered in a dose of about 0.05 to 50 mg/kg/day.

18. The method of claim 1 wherein said mammal is a human.

19. A method for identifying a compound that is a candidate for the treatment of LUTS in a mammal comprising the steps of:
(a) establishing that a test compound binds to α_{1b}- and α_{1d}-adrenergic receptors with an affinity at least about 6-fold greater than the affinity with which said compound binds to α_{1b}-adrenergic receptor; and
(b) establishing that the test compound described in step (a) is a candidate for the treatment LUTS in a mammal.

20. The method of claim 19 wherein said test compound is established to be a candidate for the treatment of LUTS by evaluating the effects of said test compound in an animal model system.

21. The method of claim 20 wherein said animal model evaluates the effect of said test compound on unstable bladder contractions.

22. The method of claim 19 wherein said mammal is a human.

23. A method of treating LUTS in a mammal in need of such treatment, comprising exposing a lower urinary tract tissue of said mammal to a therapeutically effective amount of a ligand that binds to α₁ₐ-and α_{1d}-adrenergic receptors with an affinity at least about 6-fold greater affinity than said compound binds to the α_{1b}-adrenergic receptor.

24. The method of claim 23 wherein said mammal is a human.

25. The method of claims 1 or 23 wherein the ligand is used for the treatment of irritative LUTS.

26. The method of claims 1 or 23 wherein the ligand is used to treat obstructive LUTS.

27. The method of claim 1 or 23 wherein the ligand is used for the treatment of LUTS due to BPH.

28. The method of claim 1 or 23 wherein the ligand is used to treat NLUTD.

29. The method of claim 1 or 23 wherein the ligand is administered with an anti-cholinergic agent.

30. The method of claim 1 or 23 wherein the administered ligand is a compound of formula I, 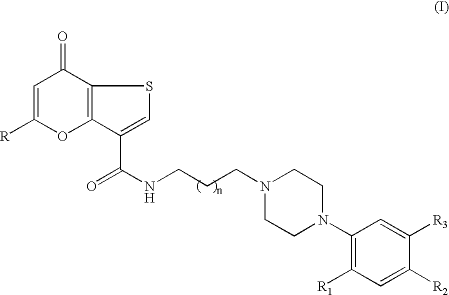
wherein
R is chosen from the group consisting of an aryl, cycloalkyl, and polyhaloalkyl group,
R₁ is chosen from the group consisting of an alkyl, alkoxy, polyfluoroalkoxy, hydroxy and trifluoromethanesulfonyloxy group, each of R₂ and R₃ being independently chosen from the group consisting of a hydrogen atom, halogen atom, alkoxy, and polyfluoroalkoxy group, and
n is 0, 1 or 2,
or a piperazine-N-oxide thereof or a pharmaceutically acceptable salt of any of the foregoing.

31. The method of claim 1 or 23 wherein the administered ligand is a compound of formula II, 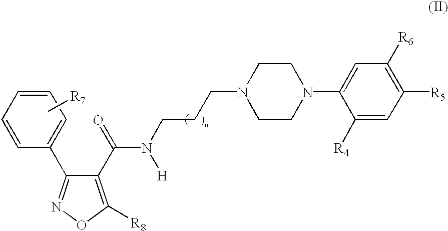
wherein
R₄ is selected from the group consisting of alkyl, alkoxy, polyfluoroalkoxy, hydroxy and trifluoromethanesulfonyloxy groups,
each of R₅ and R₆ is independently selected from the group consisting of hydrogen atom, halogen atom, polyfluoroalkoxy and alkoxy groups,
R₇ is one or more substituents selected from the group consisting of hydrogen atom, halogen atom, alkyl, alkoxy, nitro, amino, acylamino, cyano, alkoxycarbonyl, and carboxamido group,
R₈ represents a hydrogen atom or an alkyl or arylalkyl group, and
n is 0, 1 or 2, or
a piperazine-N-oxide thereof or a pharmaceutically acceptable salt of any of the foregoing.

32. The method of claim 1 or 23 wherein the administered ligand is a compound of formula III, 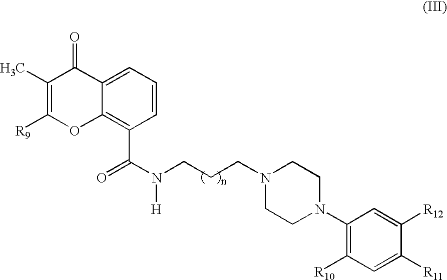
wherein
R₉ is selected from the group consisting of a phenyl, alkoxycarbonyl, alkylcarbonyl, carbamoyl, alkylcarbamoyl, dialkylcarbamoyl, cyano and alkoxycarbonylamino group,
R₁₀ is selected from the group consisting of an alkyl, alkoxy, polyfluoroalkoxy, hydroxy and trifluoromethanesulphonyloxy group,
each of R₁₁, and R₁₂ is independently selected from the group consisting of hydrogen atom, halogen atom, polyfluoroalkyl, polyfluoroalkoxy, cyano, and carbamoyl group, and
n is 0, 1 or 2,
with the proviso that if R₉ represents a phenyl group and both R₁₁ and R₁₂ represent hydrogen and/or halogen atoms, then R₁₀ represents a polyfluoroalkoxy or trifluoromethanesulphonyloxy group,
or a piperazine-N-oxide or pharmaceutically acceptable salt of such a compound.
